(19) United States
(12) Patent Application Publication
(10) Pub. No.: US 2026/0234131 A1
(43) Pub. Date: Aug. 13, 2026

(54) THERAPEUTIC ALKALOID COMPOUNDS

(71) Applicant: Sensorium Therapeutics, Inc., Boston, MA (US)

(72) Inventor: Jacob M. Hooker, Winchester, MA (US)

(21) Appl. No.: 19/470,988

(22) PCT Filed: Apr. 4, 2024
(86) PCT No.: PCT/US24/22930

§ 371 Date: Sep. 30, 2025

Related U.S. Application Data
(60) Provisional application No. 63/457,552, filed on Apr. 6, 2023.

(51) Int. Cl. C07D 405/04; A61K 31/403; A61K 31/423; C07D 409/04; C07D 411/04; C07D 413/04; C07D 493/10; C07D 495/10; C07D 497/10

(52) U.S. Cl. CPC C07D 405/04; A61K 31/403; A61K 31/423; C07D 409/04; C07D 411/04; C07D 413/04; C07D 493/10; C07D 495/10; C07D 497/10

(57) ABSTRACT

Disclosed are compounds that are derivatives of mesembrine, and related methods of preparing and using these compounds. The disclosed compounds may inhibit SERT.

### RELATED APPLICATIONS

This patent application claims the benefit of priority to U.S. Provisional Patent Application No. 63/457,552, filed Apr. 6, 2023; which is incorporated by reference in its entirety.

### TECHNICAL FIELD

The present disclosure relates to the field of medicine, including the discovery of alkaloid compounds useful for eliciting antidepressant and/or anxiolytic effects by inhibiting, in part, the serotonin transporter protein (5-HTT).

### BACKGROUND

Certain bioactive indole alkaloid compounds obtained from plants of the genus Sceletium, such as mesembrine and mesembrenone, can inhibit serotonin (5-HT) uptake and phosphodiesterase-4 (PDE-4) and have been explored as potential treatments for certain central nervous system (CNS) conditions such as mild to moderate depression.

Phosphodiesterase-4 (PDE-4) enzymes hydrolyze the cyclic nucleotide intracellular second messengers (cAMP and cGMP), leading to inactivation or inhibition of these enzymes and resulting in elevated levels of cAMP and cGMP in the cell and prolonging the action of these enzymes on downstream signaling pathways. Four isoforms of PDE-4 enzymes (designated PDE4a, PDE4b, PDE4c and PDE4d) have been identified, with the PDE4a, PDE4b and PDE4d isoforms predominantly expressed in the brain. Signaling pathways including PDE-4 are believed to be involved in diseases and disorders such as depression. Therapeutic compounds for selective inhibition of the certain PDE4 isoforms can be utilized for the treatment or prevention of depression and/or anxiety while minimizing or alleviating detrimental effects of inhibiting other PDE4 isoenzymes. One concern with the use of known PDE4 inhibitors is the side effect of emesis, which has been observed for several candidate compounds. PDE4 inhibitors, such as the brain-penetrant inhibitor rolipram, influence central function in a dose-dependent manner, consistent with their potential use in the treatment of depression and cognitive disorders in humans. However higher doses of these compounds can give rise to mechanism-related side effects such as emesis.

Natural products obtained from plants of the genus Sceletium contain varying amounts of (−) mesembrine and (+)/(−) mesembrenone. Naturally occurring (−) mesembrine from Sceletium tortuosum has been reported as having serotonin (5-HT) uptake inhibitory activity useful in treating mental health conditions, such as mild to moderate depression, and mesembrine hydrochloride has been reported to be a phosphodiesterase 4 (PDE4) inhibitor. Naturally occurring (−) mesembrenone from Sceletium tortuosum is reported as a potent selective serotonin reuptake inhibitor (Ki=27 nM) and inhibitor of a phosphodiesterase 4 (PDE4) inhibitor. Mesembrenone is a most potent inhibitor of PDE4, while mesembrine is more selective towards the serotonin receptor.

TABLE A
Summary of analysis of the concentration response curves of alkaloids on binding to
the 5-HT transporter and on activity of PDE-4B (Harvey et al., 2011)

| | 5-HT transporter (“SERT”) | | PDE-4B | |
|---|---|---|---|---|
| Compound | Ki (nM) | nH | IC₅₀ (nM) | nH |
| Mesembrine | 1.4 | 1.0 | 7,800 | 1.3 |
| Mesembrenone | 27 | 1.0 | 470 | 0.8 |

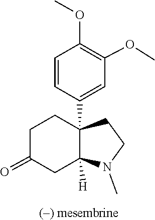 [SMILES: COc1ccc([C@@]23CCC(=O)C[C@@H]2N(C)CC3)cc1OC]

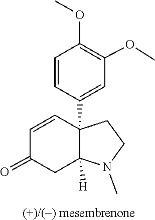 [SMILES: COc1ccc([C@@]23C=CC(=O)C[C@@H]2N(C)CC3)cc1OC]

The therapeutic use of mesembrine and mesembrenone has been limited by the variability and instability of the content of the compounds in natural extract products, and the instability and pharmacokinetic profile of these compounds as obtained from natural products.

There remains an unmet need for new compounds inhibiting the serotonin transporter protein (5-HTT).

### SUMMARY

Described are compounds of Formula (I):

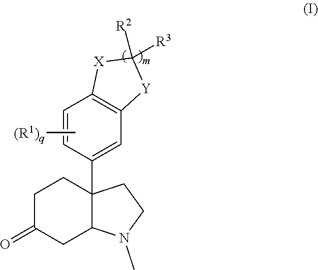

or a pharmaceutically acceptable salt thereof;
wherein
each of X and Y is independently selected from O, NR⁴, and S;
each R¹ is independently deuterium, halo, alkyl, alkenyl, alkynyl, cycloalkyl, cycloalkenyl, heterocycloalkyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocycloalkyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H or alkyl; wherein each hydrogen atom in alkyl is optionally substituted by halo, deuterium, cycloalkyl, or OR^{a}; or R² and R³ taken together with the atoms to which they are attached combine to form carbonyl, cycloalkyl, or heterocyclyl; wherein each hydrogen atom in cycloalkyl and heterocyclyl is optionally substituted by halo or OR^{a};
R⁴ is H or alkyl;
m is 1 or 2;
q is 0, 1, 2, or 3;
each R^{a} and R^{b} is independently H, alkyl, alkenyl, cycloalkyl, heterocycloalkyl, aryl, or heteroaryl; or if an instance of R¹ is —NR^{a}R^{b}, then R^{a} and R^{b} may combine with the nitrogen atom to which they are attached to form heterocycloalkyl or heteroaryl, wherein each hydrogen atom in alkyl, alkenyl, cycloalkyl, heterocycloalkyl, aryl, or heteroaryl is optionally substituted by halo, hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl.

In certain embodiments, the compound is selected from:

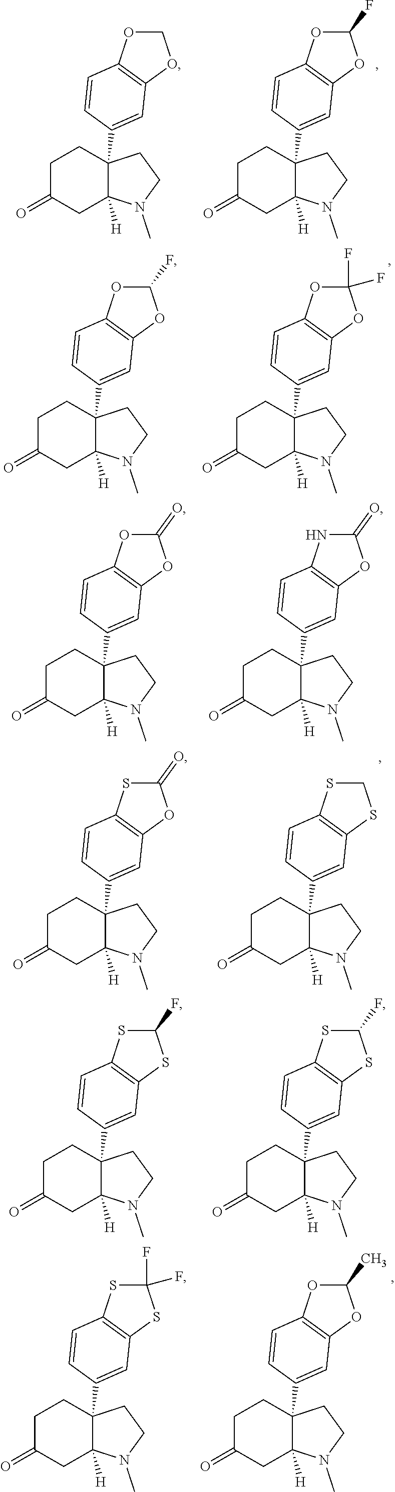

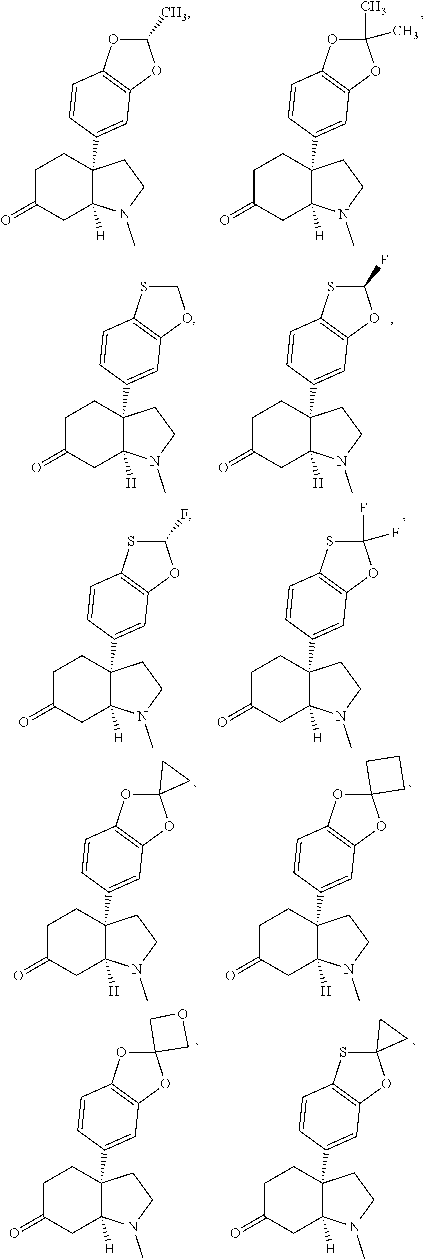

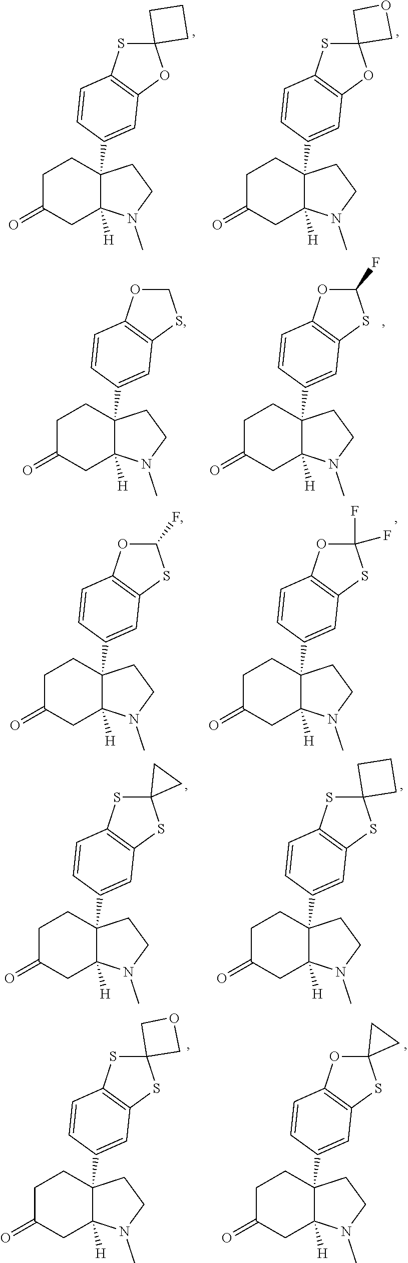

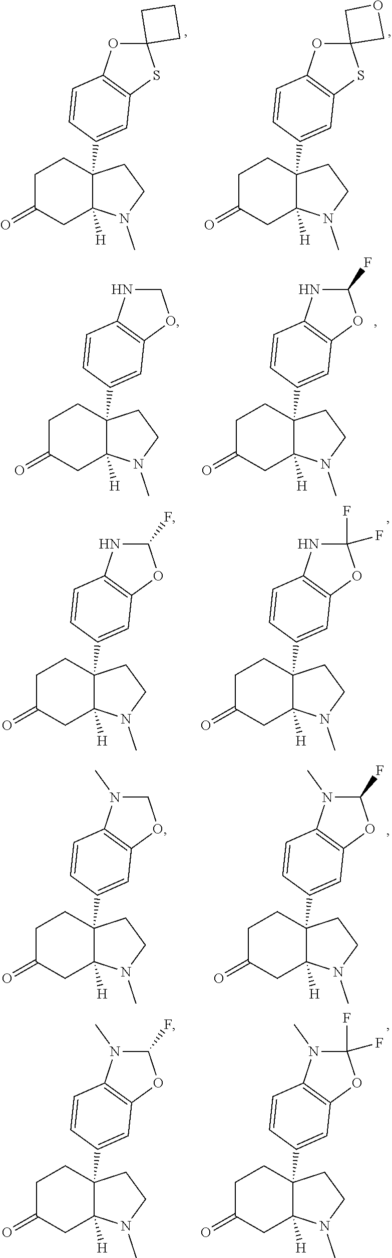

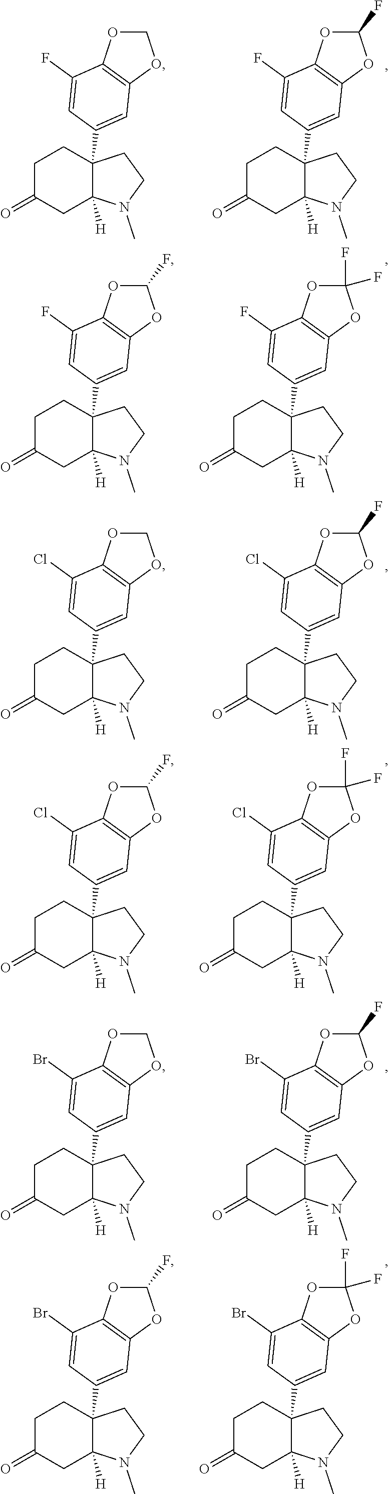

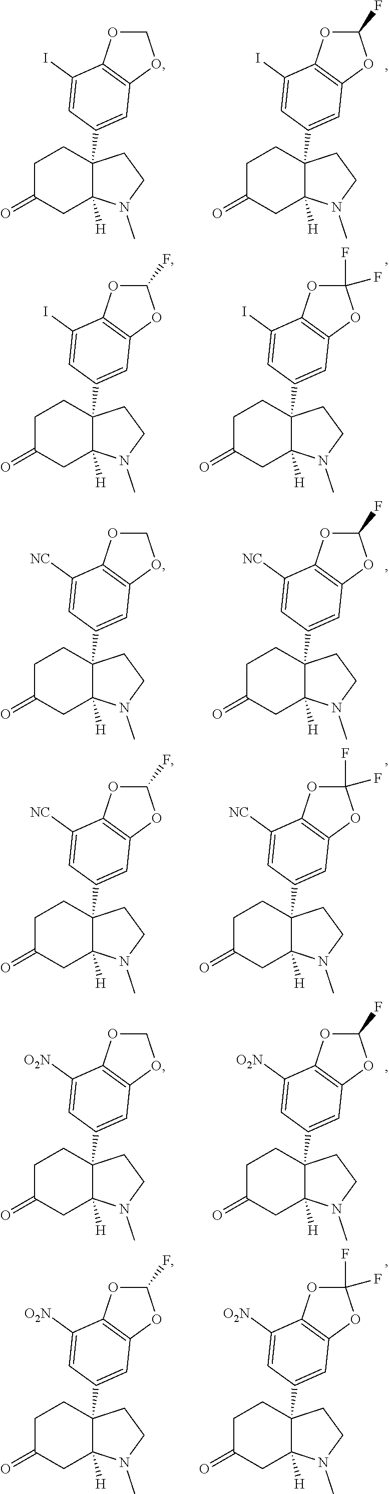

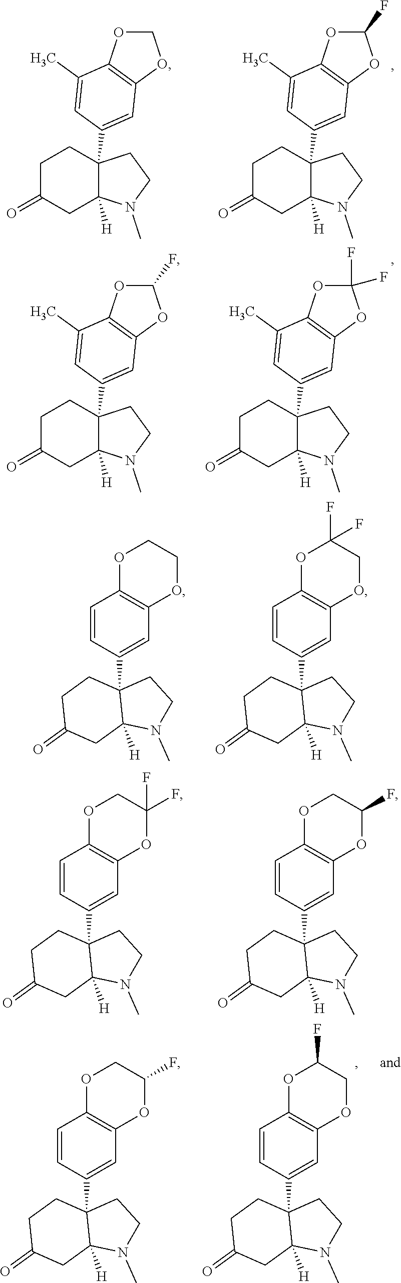

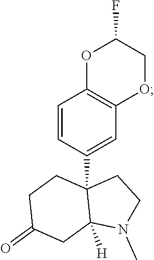 [SMILES: CN1CC[C@]2(c3ccc4c(c3)OC[C@@H](F)O4)CCC(=O)C[C@H]12]

or a pharmaceutically acceptable salt thereof.

### DETAILED DESCRIPTION

The present invention is based, at least in part, on analogs of mesembrine. Although (−) mesembrine is bioactive with certain desirable pharmacologic effects, certain other properties are less than ideal for use as a therapeutic. For example, the pharmacokinetics described for (−) mesembrine show rapid metabolism and excretion, which an undesirably low half-life in plasma of less than 2 hours. To take advantage of the desirable properties of mesembrine, compounds have been developed and described here.

### Compounds of the Invention

In certain embodiments, the compound is of Formula (I):

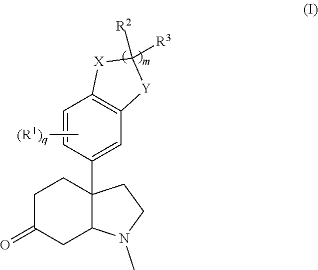

or a pharmaceutically acceptable salt thereof;
wherein
each of X and Y is independently selected from O, NR⁴, and S;
each R¹ is independently deuterium, halo, alkyl, alkenyl, alkynyl, cycloalkyl, cycloalkenyl, heterocycloalkyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocycloalkyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H or alkyl; wherein each hydrogen atom in alkyl is optionally substituted by halo, deuterium, cycloalkyl, or OR^{a}; or R² and R³ taken together with the atoms to which they are attached combine to form carbonyl, cycloalkyl, or heterocyclyl; wherein each hydrogen atom in cycloalkyl and heterocyclyl is optionally substituted by halo or OR^{a};
R⁴ is H or alkyl;
m is 1 or 2;
q is 0, 1, 2, or 3;
each R^{a} and R^{b} is independently H, alkyl, alkenyl, cycloalkyl, heterocycloalkyl, aryl, or heteroaryl; or if an instance of R¹ is —NR^{a}R^{b}, then R^{a} and R^{b} may combine with the nitrogen atom to which they are attached to form heterocycloalkyl or heteroaryl, wherein each hydrogen atom in alkyl, alkenyl, cycloalkyl, heterocycloalkyl, aryl, or heteroaryl is optionally substituted by halo, hydroxy, alkyl, alkanol, aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, alkyl, alkenyl, cycloalkyl, heterocyclyl, aryl, or heteroaryl.

In certain embodiments, the compound is of Formula (IA):

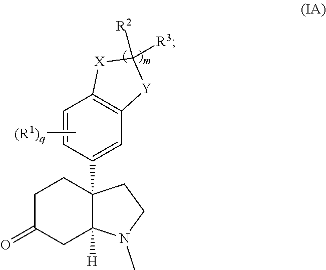

or a pharmaceutically acceptable salt thereof. In certain embodiments, the compound has the absolute stereochemistry shown.

In certain embodiments, the compound is of Formula (II):

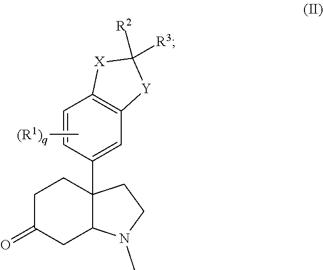

or a pharmaceutically acceptable salt thereof.

In certain embodiments, the compound is of Formula (IIA):

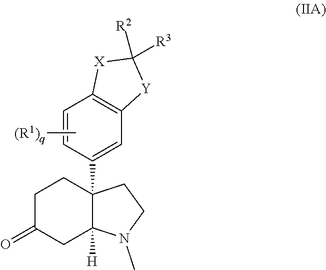

or a pharmaceutically acceptable salt thereof. In certain embodiments, the compound has the absolute stereochemistry shown.

In certain embodiments, q is 1, 2, or 3. In certain embodiments, q is 1. In certain embodiments, q is 0.

In certain embodiments, the compound is of Formula (I-1):

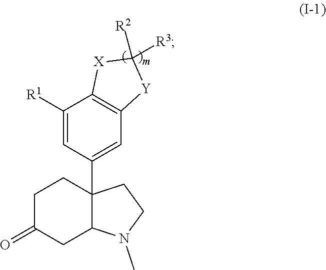

or a pharmaceutically acceptable salt thereof.

In certain embodiments, the compound is of Formula (IA-1):

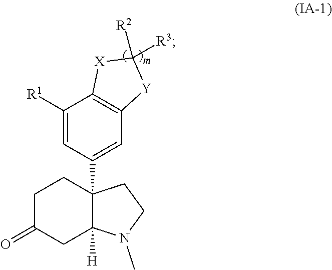

or a pharmaceutically acceptable salt thereof. In certain embodiments, the compound has the absolute stereochemistry as shown.

In certain embodiments, the compound is of Formula (II-1):

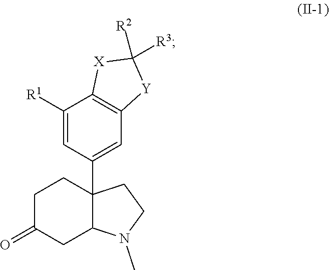

or a pharmaceutically acceptable salt thereof.

In certain embodiments, the compound is of Formula (IIA-1):

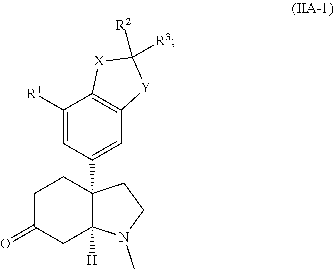

or a pharmaceutically acceptable salt thereof. In certain embodiments, the compound has the absolute stereochemistry as shown.

In certain embodiments, each of R² and R³ is H. In some embodiments, one of R² and R³ is halo or alkyl. In some embodiments, one of R² and R³ is fluoro, chloro, bromo, or iodo. In some embodiments, one of R² and R³ is C₁-C₆ alkyl, for example, methyl.

In certain embodiments, each of R² and R³ is independently halo or alkyl. In some embodiments, each of R² and R³ can be independently selected from fluoro, chloro, bromo, and iodo. In some embodiments, each of R² and R³ is independently C₁-C₆ alkyl, for example methyl.

In certain embodiments, R² and R³ are taken together with the carbon to which they are attached combine to form carbonyl, cycloalkyl, or heterocyclyl. In some embodiments, in one instance, R² and R³ taken together with the carbon to which they are attached combine to form C₃-C₆ cycloalkyl (e.g., in compounds where the ring containing X and Y is a six-membered ring such that there are R²s and R³s on adjacent carbons, only a single R² and single R³ combine with the carbon atom to which they are attached form C₃-C₆ cycloalkyl). In some embodiments, in one instance, R² and R³ taken together with the carbon to which they are attached combine to form C₃-C₄ cycloalkyl. In some embodiments, in one instance, R² and R³ taken together with the carbon to which they are attached combine to form 3- to 6-membered heterocyclyl. In some embodiments, in one instance, R² and R³ taken together with the carbon to which they are attached combine to form carbonyl.

In certain embodiments, the compound is of Formula (III):

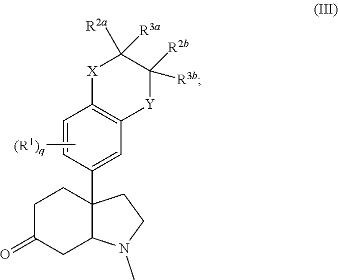

or a pharmaceutically acceptable salt thereof; wherein
each of R^{2a}, R^{3a}, R^{2b} and R^{3b} is independently H or alkyl; wherein each hydrogen atom in alkyl is optionally substituted by halo, deuterium, cycloalkyl, or OR^{a}; or R^{2a} and R^{3a} or R^{2b} and R^{3b} taken together with the atoms to which they are attached combine to form carbonyl, cycloalkyl, or heterocyclyl; wherein each hydrogen atom in cycloalkyl and heterocyclyl is optionally substituted by halo or OR^{a}.

In certain embodiments, the compound is of Formula (IIIA):

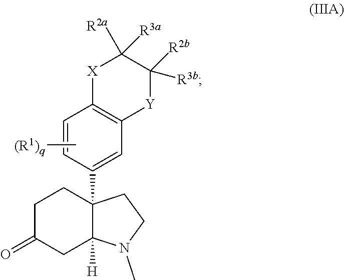

or a pharmaceutically acceptable salt thereof. In certain embodiments, the compound has the absolute stereochemistry shown.

In certain embodiments, the compound is of Formula (III-1):

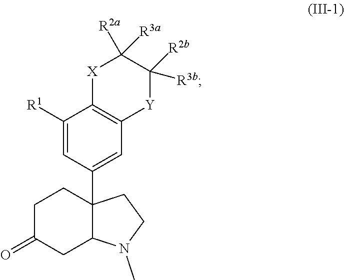

or a pharmaceutically acceptable salt thereof.

In certain embodiments, the compound is of Formula (IIIA-1):

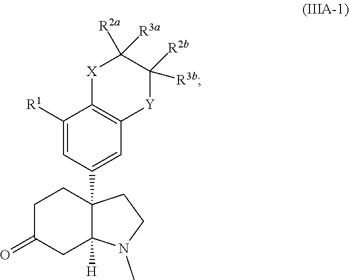

or a pharmaceutically acceptable salt thereof. In certain embodiments, the compound has the absolute stereochemistry shown.

In certain embodiments, each of R^{2a}, R^{3a}, R^{2b} and R^{3b} is H. In some embodiments, one of R^{2a}, R^{3a}, R^{2b} and R^{3b} is halo or alkyl. In some embodiments, one of R^{2a}, R^{3a}, R^{2b} and R^{3b} is fluoro, chloro, bromo, or iodo. In some embodiments, one of R^{2a}, R^{3a}, R^{2b} and R^{3b} is C₁-C₆ alkyl. In some embodiments, one of R^{2a}, R^{3a}, R^{2b} and R^{3b} is methyl. In some embodiments, two of R^{2a}, R^{3a}, R^{2b} and R^{3b} are halo or alkyl. In some embodiments, two of R², R^{3a}, R^{2b} and R^{3b} are independently fluoro, chloro, bromo, or iodo. In some embodiments, two of R^{2a}, R^{3a}, R^{2b} and R^{3b} are independently C₁-C₆ alkyl. In some embodiments, two of R^{2a}, R^{3a}, R^{2b} and R^{3b} are methyl. In some embodiments, R^{2a} and R^{3a} or R^{2b} and R^{3b} taken together with the carbon to which they are attached combine to form carbonyl, cycloalkyl, or heterocyclyl. In some embodiments, R^{2a} and R^{3a} or R^{2b} and R^{3b} taken together with the carbon to which they are attached combine to form C₃-C₆ cycloalkyl. In some embodiments, R^{2a} and R^{3a} or R^{2b} and R^{3b} taken together with the carbon to which they are attached combine to form C₃-C₄ cycloalkyl. In some embodiments, R^{2a} and R^{3a} or R^{2b} and R^{3b} taken together with the carbon to which they are attached combine to form 3- to 6-membered heterocyclyl.

In certain embodiments, each R¹ is independently halo, alkyl, cyano, or nitro. In some embodiments, each R¹ is independently fluoro, chloro, or bromo. In some embodiments, each R¹ is independently C₁-C₆ alkyl. In some embodiments, each R¹ is methyl. In some embodiments, each R¹ is cyano. In some embodiments, each R¹ is nitro.

In certain embodiments, at least one of X and Y is O. In some embodiments, X is O. In some embodiments, Y is O. In some embodiments, at least one of X and Y is S. In some embodiments, X is S. In some embodiments, Y is S.

In certain embodiments, X is NR⁴. In certain embodiments, R⁴ is C₁-C₆ alkyl.

In certain embodiments, the compound is of Formula (IIA-1) or Formula (III-1):

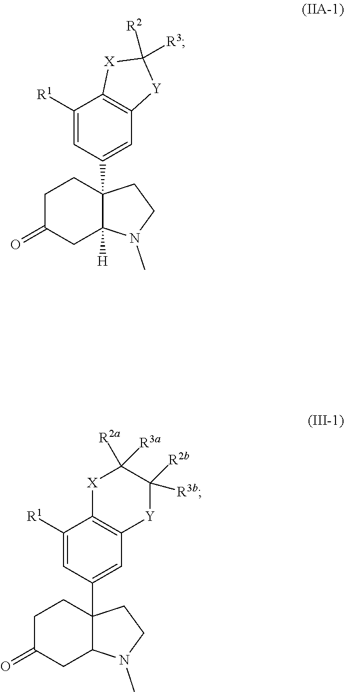

or a pharmaceutically acceptable salt thereof; wherein:
each of X and Y is independently selected from O, NR⁴ and S;
each R¹ is independently hydrogen, deuterium, halo, alkyl, alkenyl, alkynyl, cycloalkyl, cycloalkenyl, heterocycloalkyl, aryl, heteroaryl, —OR^{a}, —NR^{a}R^{b}, —CHO, —C(O)R^{a}, —CO₂R^{a}, —C(O)NR^{a}R^{b}, —CN, or nitro; wherein each hydrogen atom in alkyl, alkenyl, alkynyl, cycloalkyl, cycloalkenyl, heterocycloalkyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, or nitro;
each of R² and R³ is independently H or alkyl; wherein each hydrogen atom in alkyl is optionally substituted by halo, deuterium, cycloalkyl, or OR^{a}; or R² and R³ taken together with the atoms to which they are attached combine to form carbonyl, cycloalkyl, or heterocyclyl; wherein each hydrogen atom in cycloalkyl and heterocyclyl is optionally substituted by halo or OR^{a};
each of R^{2a}, R^{3a}, R^{2b} and R^{3b} is independently H or alkyl; wherein each hydrogen atom in alkyl is optionally substituted by halo, deuterium, cycloalkyl, or OR^{a}; or R^{2a} and R^{3a} or R^{2b} and R^{3b} taken together with the atoms to which they are attached combine to form carbonyl, cycloalkyl, or heterocyclyl; wherein each hydrogen atom in cycloalkyl and heterocyclyl is optionally substituted by halo or OR^{a};
each R^{a} and R^{b} is independently H, alkyl, alkenyl, cycloalkyl, heterocycloalkyl, aryl, or heteroaryl; or if an instance of R¹ is —NR^{a}R^{b}, then R^{a} and R^{b} may combine with the nitrogen atom to which they are attached to form heterocycloalkyl or heteroaryl, wherein each hydrogen atom in alkyl, alkenyl, cycloalkyl, heterocycloalkyl, aryl, or heteroaryl is optionally substituted by halo, hydroxy, alkyl, alkanol, aryl, —OR^{c}, —NR^{c}R^{d}, —CHO, —C(O)R^{c}, —CO₂R^{c}, —C(O)NR^{c}R^{d}, —CN, or nitro.

In certain embodiments, each R¹ is independently hydrogen, deuterium, halo, C₁-C₄ alkyl, nitro, or CN. In some embodiments, each R¹ is hydrogen. In some embodiments, each R¹ is methyl. In some embodiments, each R¹ is fluoro, chloro, bromo or iodo. In some embodiments, each R¹ is fluoro. In some embodiments, each R¹ is chloro. In some embodiments, each R¹ is bromo. In some embodiments, each R¹ is iodo. In some embodiments, each R¹ is nitro. In some embodiments, each R¹ is CN.

In certain embodiments, each of R² and R³ is independently H or C₁-C₄ alkyl; wherein each hydrogen atom in alkyl is optionally substituted by halo, deuterium, C₃-C₆ cycloalkyl, or OR^{a}; or R² and R³ taken together with the atoms to which they are attached combine to form carbonyl, C₃-C₆ cycloalkyl, or 3-6 membered heterocyclyl; wherein each hydrogen atom in cycloalkyl and heterocyclyl is optionally substituted by halo or OR^{a};
each of R^{2a}, R^{3a}, R^{2b} and R^{3b} is independently H or C₁-C₄ alkyl; wherein each hydrogen atom in alkyl is optionally substituted by halo, deuterium, C₃-C₆ cycloalkyl, or OR^{a}; or R^{2a} and R^{3a} or R^{2b} and R^{3b} taken together with the atoms to which they are attached combine to form carbonyl, C₃-C₆ cycloalkyl, or 3-6 membered heterocyclyl; wherein each hydrogen atom in cycloalkyl and heterocyclyl is optionally substituted by halo or OR^{a}; and
each R^{a} is independently H, or C₁-C₄ alkyl; wherein each hydrogen atom in alkyl is optionally substituted by halo, hydroxy, —CN, or nitro.

In certain embodiments, each of R² and R³ is independently H; and each of R^{2a}, R^{3a}, R^{2b} and R^{3b} is H.

In certain embodiments, the compound is a compound of Formula (IIA-1). In some embodiments, each of R² and R³ is independently halo or H. In some embodiments, each of R² and R³ is independently fluoro or H. In some embodiments, each of R² and R³ is H. In some embodiments, each of R² and R³ is fluoro. In some embodiments, R² and R³ taken together with the carbon atom to which they are attached combine to form carbonyl. In some embodiments, each of R² and R³ is independently H or C₁-C₄ alkyl. In some embodiments, each of R² and R³ is independently H or methyl. In some embodiments, R² is methyl and R³ is H or methyl. In some embodiments, each of R² and R³ is independently H or methyl. In some embodiments, R² and R³ taken together with the atoms to which they are attached combine to form C₃-C₆ cycloalkyl or 3-6 membered heterocyclyl. In some embodiments, R² and R³ taken together with the atoms to which they are attached combine to form cyclopropyl. In some embodiments, R² and R³ taken together with the atoms to which they are attached combine to form cyclobutyl. In some embodiments, R² and R³ taken together with the atoms to which they are attached combine to form oxetanyl.

In certain embodiments, the compound is a compound of Formula (III-1). In some embodiments, each of R^{2a}, R^{3a}, R^{2b} and R^{3b} is independently H, halo or C₁-C₄ alkyl; wherein each hydrogen atom in alkyl is optionally substituted by halo. In some embodiments, each of R^{2a}, R^{3a}, R^{2b} and R^{3b} is independently H, fluoro or methyl.

In certain embodiments, X is O. In certain embodiments, X is NR⁴. In certain embodiments, X is S. In certain embodiments, Y is O. In certain embodiments, wherein Y is NR⁴. In certain embodiments, Y is S. In certain embodiments, R⁴ is H. In certain embodiments, R⁴ is methyl.

In certain embodiments, wherein R^{a} is methyl. In certain embodiments, R^{a} is H.

In certain embodiments, R^{b} is H. In certain embodiments, R^{b} is methyl.

In certain embodiments, R^{c} is H. In certain embodiments, R^{c} is methyl.

In certain embodiments, R^{d} is H. In certain embodiments, R^{d} is methyl.

In certain embodiments, the compound is selected from:

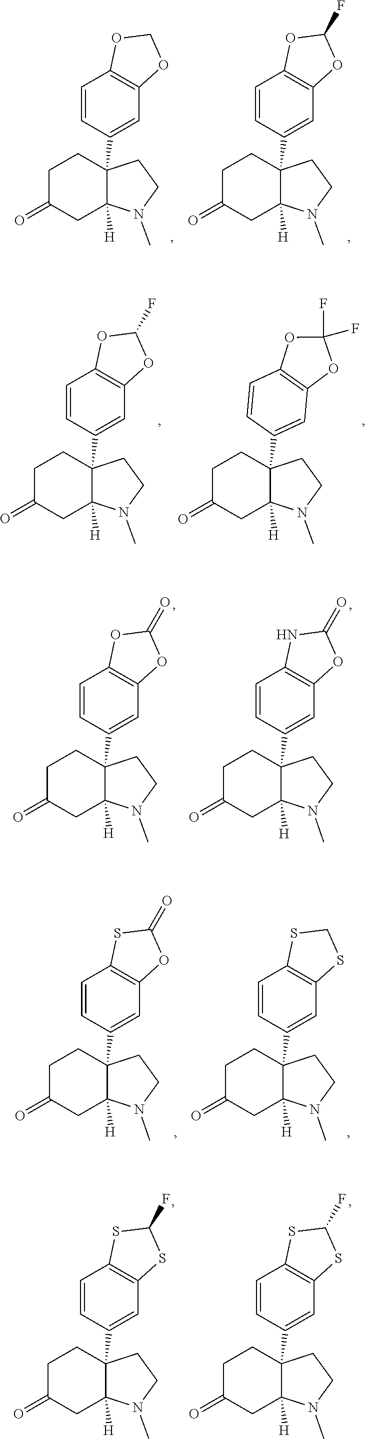

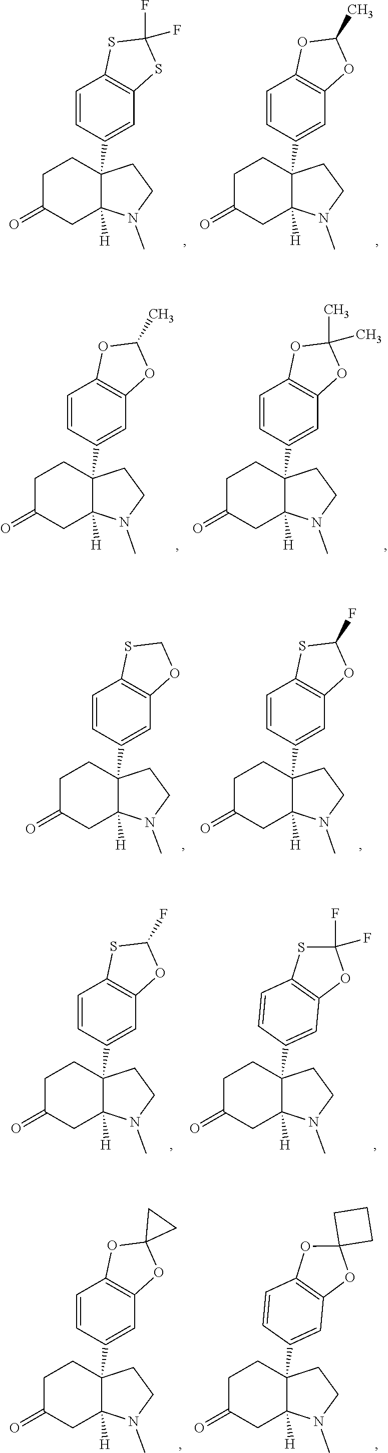

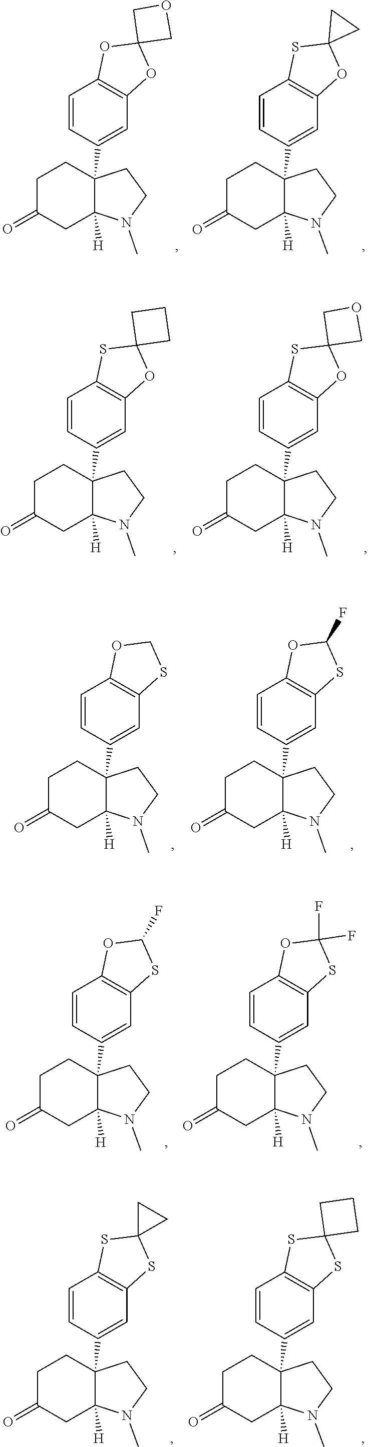

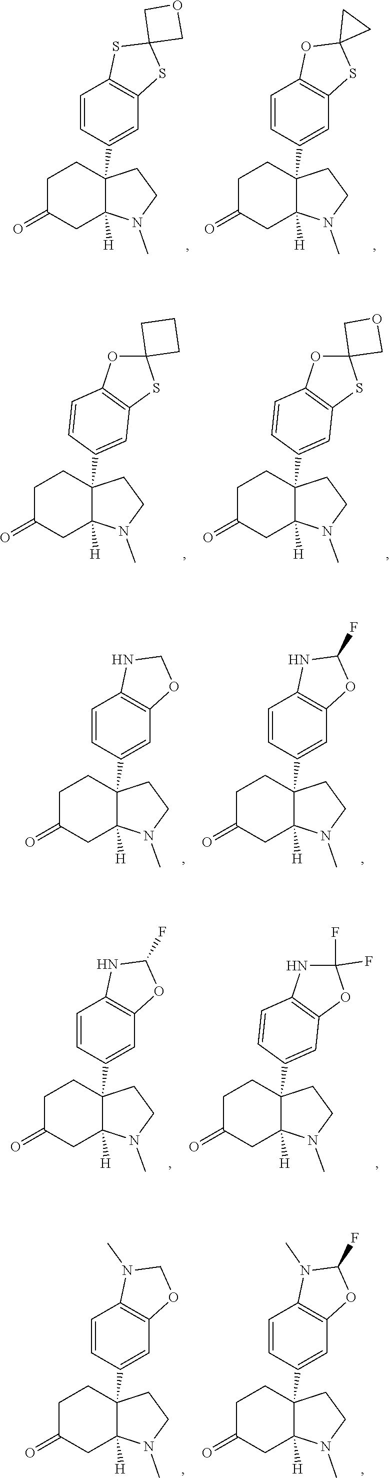

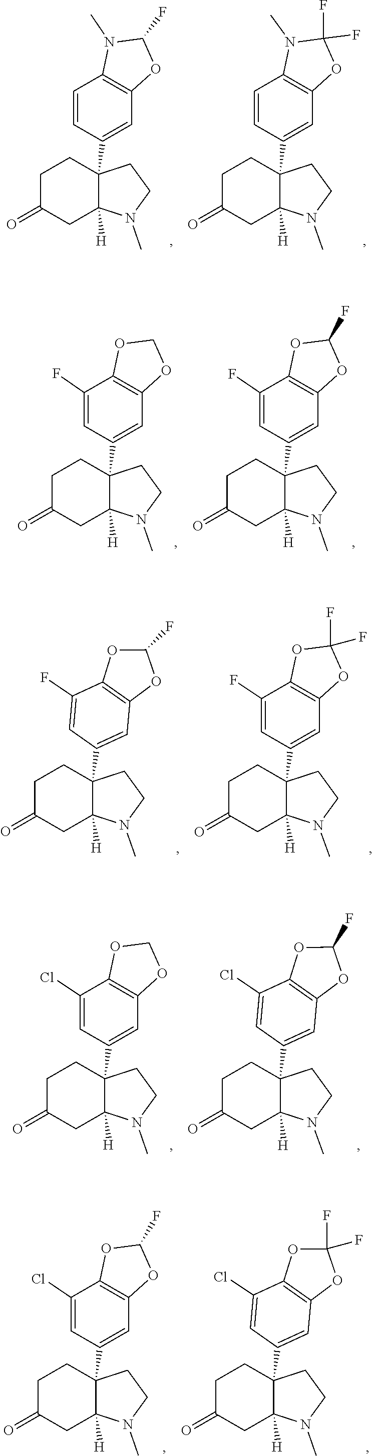

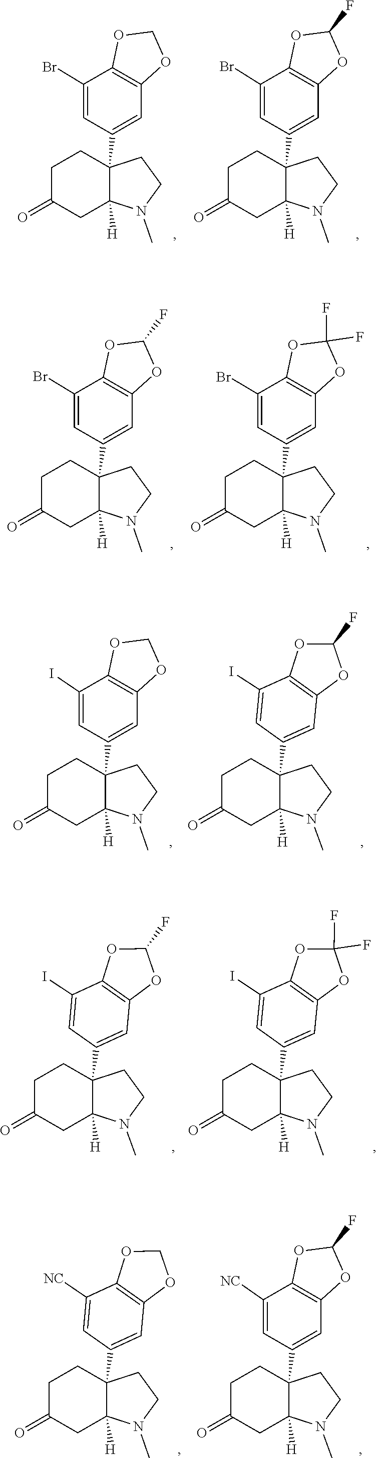

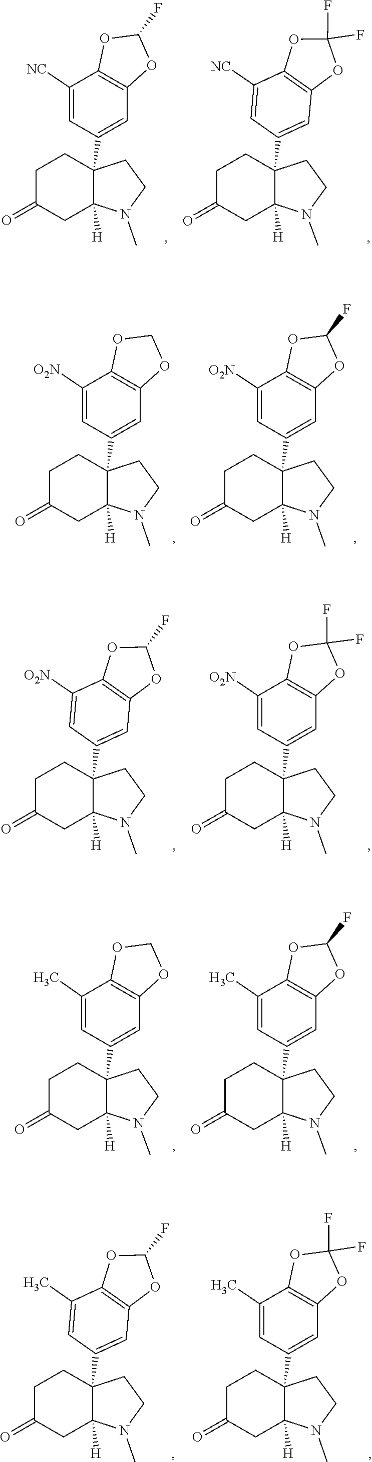

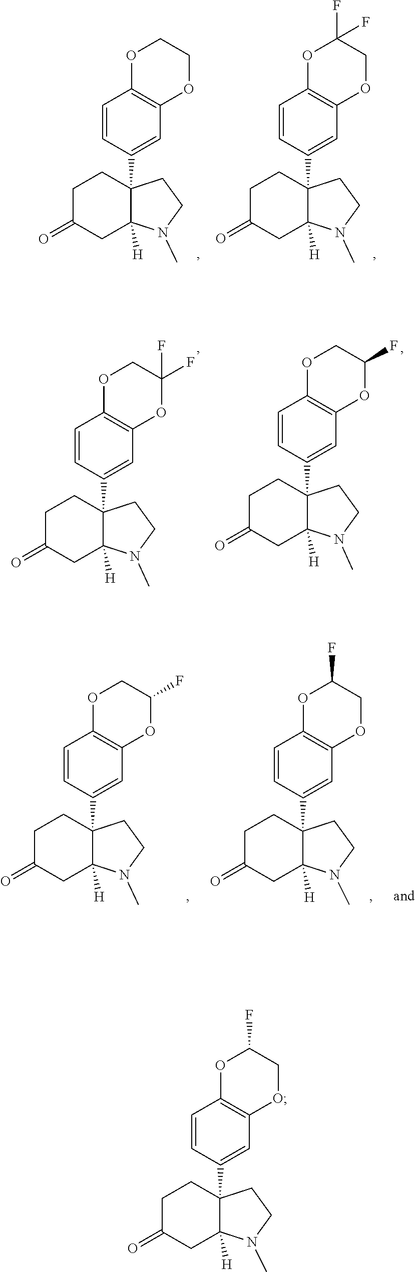

or a pharmaceutically acceptable salt thereof. In certain embodiments, the compound has the absolute stereochemistry shown.

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g., intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In some embodiments, pharmaceutical compositions comprising compounds of Formula (I) or pharmaceutically acceptable salts thereof can be formulated for oral administration. For example, a compound provided herein can be combined with suitable compendial excipients to form an oral unit dosage form, such as a capsule or tablet, containing a target dose of a compound of Formula (I). The drug product can be prepared by first manufacturing the compound of Formula (I) as an active pharmaceutical ingredient (API), followed by roller compaction/milling with intragranular excipients and blending with extra granular excipients. A Drug Product can contain the selected compound of Formula (I) as the API and excipient components in a tablet in a desired dosage strength. The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include a compound of Formula (I) as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present invention may contain conventional pharmaceutical carriers and/or auxiliary agents. In some embodiments, he pharmaceutical compositions according to the present invention may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

Liquid dosage forms useful for oral administration include pharmaceutically acceptable carriers and the active ingredient provided as a solid form for reconstitution prior to administration or as a liquid (e.g., solutions, suspensions, or emulsions). In addition to the active ingredient, a liquid dosage forms may contain inert diluents commonly used in the art. For example, formulations of pharmaceutically acceptable compositions for injection can include aqueous solutions such as water or physiologically buffered saline or other solvents or vehicles suitable for the intended route of administration. In some embodiments, the pharmaceutical composition is formulated for parenteral administration.

The therapeutically effective amount of a pharmaceutical composition can be determined by human clinical trials to determine the safe and effective dose for a patient with a relevant diagnosis. It is generally understood that the effective amount of the compound may vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the pharmaceutical composition at a dose and dose interval determined to be safe and effective for the patient.

The present disclosure includes the use of pharmaceutically acceptable salts of compounds of the invention in the compositions and methods of the present invention. Pharmaceutically acceptable salts include, for example, acid-addition salts and base-addition salts. The acid that is added to a compound to form an acid-addition salt can be an organic acid or an inorganic acid. A base that is added to a compound to form a base-addition salt can be an organic base or an inorganic base. In some embodiments, a pharmaceutically acceptable salt is a metal salt, in some embodiments, a pharmaceutically acceptable salt is an ammonium salt. For example, a pharmaceutically acceptable acid addition salt can exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

In some embodiments, a compound of Formula (I) can provide additional beneficial properties. For example, the compounds described herein may provide beneficial therapeutic properties while minimizing emesis. For example, compounds of Formula (I) may have improved selectivity for inhibiting PDE4 and the specific variants thereof. In some embodiments, the compounds of Formula (I) described herein inhibit specific variants of PDE4.

### Methods of Treatment

Methods of using compounds disclosed herein are also provided. The disclosure also includes pharmaceutical compositions comprising one or more SERT inhibiting compounds, as described herein, or a pharmaceutically acceptable salt thereof, and a pharmaceutically acceptable excipient. In some embodiments, pharmaceutical compositions reported herein can be provided in a unit dosage form (e.g., capsule, tablet or the like). Pharmaceutical compositions comprising a compound of the invention can be provided in an oral dosage form such as a capsule or tablet. The oral dosage form optionally comprises one or more fillers, disintegrants, lubricants, glidants, anti-adherents and/or anti-statics. In some embodiments, an oral dosage form is prepared via dry blending. In some embodiments, an oral dosage form is a tablet and is prepared via dry granulation. For example, a compound of the present disclosure can be formulated as a test article for evaluation in animal models and (if appropriate) subsequent human clinical trials to determine a therapeutically effective dose and dose frequency for humans. The pharmaceutical compositions may be orally administered in any orally acceptable dosage form. Accordingly, a patient and/or subject can be selected for treatment using a compound described herein by first evaluating the patient and/or subject to determine whether the subject is in need of inhibition of SERT, and if the subject is determined to be in need of inhibition of SERT, then administering to the subject a pharmaceutical composition comprising one or more compounds described herein, or pharmaceutically acceptable salts thereof.

The compounds described herein may be administered to treat CNS disorders and/or inflammatory conditions. Exemplary CNS disorders include generalized anxiety, acute anxiety and panic attacks, social anxiety, panic disorders, major depressive disorder, cognitive disorders, including Alzheimer's disease and other neurodegenerative disorders, neurodevelopmental disorders, schizophrenia, bipolar disorder, obsessive-compulsive disorder, multiple sclerosis, attention deficit-hyperactivity disorder, Bulimia nervosa, Huntington's disease, stroke, autism, premenstrual dysphoric disorder. Exemplary inflammatory conditions include chronic obstructive pulmonary disease (COPD), asthma and rheumatoid arthritis. In some embodiments, methods of treating a patient suffering from a disease comprise administering to a patient a composition comprising a compound disclosed herein for the treatment or prevention of a mental health disorder. In some embodiments, methods of treating a patient suffering from a disease comprise administering to a patient a composition comprising a compound disclosed herein for the treatment or prevention of a diagnosed condition selected from anxiety and depression. In some embodiments, the compound disclosed herein is administered to the patient in a unit dose. In some embodiments, a method comprises the administration to a patient in need thereof of a therapeutically effective amount of a compound of Formula (I) for the treatment of a disease selected from the group consisting of mild to moderate depression and major depressive episodes. In some embodiments, a method comprises the administration to a patient in need thereof of a therapeutically effective amount of a compound of Formula (I) for the treatment of anxiety. In some embodiments, a method comprises the administration to a patient in need thereof of a therapeutically effective amount of a compound of Formula (I) for the treatment of depression. In some embodiments, a method comprises the administration to a patient in need thereof of a therapeutically effective amount of a compound of Formula (I) for the treatment of a condition selected from the group consisting of: anxiety associated with depression, anxiety with depression, mixed anxiety and depressive disorder. In some embodiments, a method comprises the administration to a patient in need thereof of a therapeutically effective amount of a compound of Formula (I) for the treatment of anxiety and hysteria or anxiety and depression.

The present disclosure provides methods of treating subjects diagnosed with various neurological and psychiatric disorders by administering to said subjects a dose of a pharmaceutical composition comprising a compound provided herein. Said disorders include, without limitation, attention deficit disorder hyperactivity disorder (ADHD), cognition impairment, anxiety disorders, especially generalized anxiety disorder (GAD), panic disorder, bipolar disorder, also known as manic depression or manic-depressive disorder, obsessive compulsive disorder (OCD), posttraumatic stress disorder (PTSD), acute stress disorder, social phobia, simple phobia, pre-menstrual dysphoric disorder (PMDD), social anxiety disorder (SAD), major depressive disorder (MDD), supranuclear palsy, eating disorders, especially obesity, anorexia nervosa, bulimia nervosa, and binge eating disorder, analgesia (including neuropathic pain, especially diabetic neuropathy), substance abuse disorders (including chemical dependencies) like nicotine addiction, cocaine addiction, alcohol and amphetamine addiction, Lesch-Nyhan syndrome, neurodegenerative diseases like Parkinson's disease, late luteal phase syndrome or narcolepsy, psychiatric symptoms anger such as, rejection sensitivity, movement disorders, like extrapyramidal syndrome, Tic disorders and restless leg syndrome (RLS), tardive dyskinesia, supranuclear palsy, sleep related eating disorder (SRED), night eating syndrome (NES), urinary incontinence (including stress urinary incontinence (SUI) and mixed incontinence), migraine, fibromyalgia syndrome (FS), chronic fatigue syndrome (CFS), sexual dysfunction especially premature ejaculation and male impotence, thermoregulatory disorders (e.g., hot flashes that may be associated with menopause), and lower back pain.

In some embodiments, methods of treating a disease or disorder comprise the administration of a therapeutically effective amount of a compound disclosed herein, wherein the disease or disorder is selected from the group consisting of major depressive disorder, social anxiety disorder, obsessive compulsive disorder (OCD), panic disorder (PD), generalized anxiety disorder (GAD), posttraumatic stress disorder (PTSD), bulimia nervosa, premenstrual dysphoric disorder (PMDD), premature ejaculation, arthritis, chronic fatigue, multiple sclerosis, lupus, irritable bowel syndrome (IBS), migraine headache, diabetic neuropathy, fibromyalgia, attention-deficit/hyperactivity disorder (ADHD), autistic spectrum disorders, bipolar depression, attention deficit disorder, chronic pain, neurocardiogenic syncope, post-traumatic stress disorders, obsessive compulsive disorders, anxiety, panic attacks, pain, neuralgic pain, postherpetic neuralgia, phobias of various types, and eating disorders.

In some embodiments, methods of treating a disease or disorder comprise the administration of a therapeutically effective amount of a compound disclosed herein, wherein the disease or disorder is selected from the group consisting of lower back pain, attention deficit hyperactivity disorder (ADHD), cognition impairment, anxiety disorders, generalized anxiety disorder (GAD), panic disorder, bipolar disorder or manic depression or manic-depressive disorder, obsessive compulsive disorder (OCD), posttraumatic stress disorder (PTSD), acute stress disorder, social phobia, simple phobias, pre-menstrual dysphoric disorder (PMDD), social anxiety disorder (SAD), major depressive disorder (MDD), postnatal depression, dysthymia, depression associated with Alzheimer's disease, Parkinson's disease, or psychosis, supranuclear palsy, eating disorders, obesity, anorexia nervosa, bulimia nervosa, binge eating disorder, analgesia, substance abuse disorders, chemical dependencies, nicotine addiction, cocaine addiction, alcohol and amphetamine addiction, Lesch-Nyhan syndrome, neurodegenerative diseases, Parkinson's disease, late luteal phase syndrome or narcolepsy, psychiatric symptoms, anger, rejection sensitivity, movement disorders, extrapyramidal syndrome, Tic disorders, restless leg syndrome (RLS), tardive dyskinesia, supranuclear palsy, sleep related eating disorder (SRED), night eating syndrome (NES), stress urinary incontinence (SUI), migraine, neuropathic pain, diabetic neuropathy, fibromyalgia syndrome (FS), chronic fatigue syndrome (CFS), sexual dysfunction, premature ejaculation, male impotence, and thermoregulatory disorders.

### Pharmaceutical Compositions

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g. intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In some embodiments, pharmaceutical compositions comprising compounds of Formula (I) or pharmaceutically acceptable salts thereof can be formulated for oral administration. For example, a compound provided herein can be combined with suitable compendial excipients to form an oral unit dosage form, such as a capsule or tablet, containing a target dose of a compound of Formula (I). The drug product can be prepared by first manufacturing the compound of Formula (I) as an active pharmaceutical ingredient (API), followed by roller compaction/milling with intragranular excipients and blending with extra granular excipients. A Drug Product can contain the selected compound of Formula (I) as the API and excipient components in a tablet in a desired dosage strength of a compound of Formula (1). The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include a compound of Formula (I) as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present invention may contain conventional pharmaceutical carriers and/or auxiliary agents. In some embodiments, he pharmaceutical compositions according to the present invention may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

Liquid dosage forms useful for oral administration include pharmaceutically acceptable carriers and the active ingredient provided as a solid form for reconstitution prior to administration or as a liquid (e.g., solutions, suspensions, or emulsions). In addition to the active ingredient, a liquid dosage forms may contain inert diluents commonly used in the art. For example, formulations of pharmaceutically acceptable compositions for injection can include aqueous solutions such as water or physiologically buffered saline or other solvents or vehicles suitable for the intended route of administration. In some embodiments, the pharmaceutical composition is formulated for parenteral administration.

The therapeutically effective amount of a pharmaceutical composition can be determined by human clinical trials to determine the safe and effective dose for a patient with a relevant diagnosis. It is generally understood that the effective amount of the compound may vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the pharmaceutical composition at a dose and dose interval determined to be safe and effective for the patient.

The present disclosure includes the use of pharmaceutically acceptable salts of compounds of the invention in the compositions and methods of the present invention.

Pharmaceutically acceptable salts include, for example, acid-addition salts and base addition salts. The acid that is added to a compound to form an acid-addition salt can be an organic acid or an inorganic acid. A base that is added to a compound to form a base addition salt can be an organic base or an inorganic base. In some embodiments, a pharmaceutically acceptable salt is a metal salt, in some embodiments, a pharmaceutically acceptable salt is an ammonium salt. For example, a pharmaceutically acceptable acid addition salt can exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g., “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups, C₁-C₁₀ branched-chain alkyl groups, cycloalkyl (alicyclic) groups, alkyl-substituted cycloalkyl groups, and cycloalkyl-substituted alkyl groups. Preferably, the “alkyl” group refers to C₁-C₇ straight-chain alkyl groups or C₁-C₇ branched-chain alkyl groups. Most preferably, the “alkyl” group refers to C₁-C₃ straight-chain alkyl groups or C₁-C₃ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “haloalkyl” refers to an alkyl group substituted with at least one hydrogen atom on a carbon replaced by a halogen. Illustrative halogens include fluoro, chloro, bromo, and iodo. Illustrative haloalkyl groups include trifluoromethyl and 2,2,2-trifluoroethyl, etc.

The term “alkoxyalkyl” refers to an alkyl group substituted with an alkoxy group and may be represented by the general formula alkyl-O-alkyl.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide” or “amido”, as used herein, refers to a group

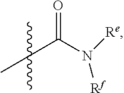

wherein R^{c} and R^{f} each independently represent a hydrogen or hydrocarbyl group, or R^{c} and R^{f} taken together with the N atom to which they are attached complete a heterocycle having from 4 to 8 atoms in the ring structure.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Preferably, the “alkoxy” group refers to C₁-C₇ straight-chain alkoxy groups or C₁-C₇ branched-chain alkoxy groups. Representative alkoxy groups include methoxy, ethoxy, propoxy, tert-butoxy and the like.

The terms “amine” and “amino” are art-recognized and refer to both unsubstituted and substituted amines and salts thereof, e.g., a moiety that can be represented by

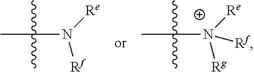

wherein R^{c}, R^{f}, and R^{g}, each independently represent a hydrogen or a hydrocarbyl group, or R and R taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring, for example a phenyl. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

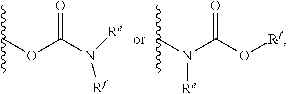

wherein R^{c} and R^{f} independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹, wherein R⁹ represents a hydrocarbyl group.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains six or fewer carbon atoms, preferably four or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

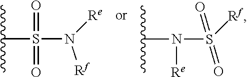

wherein R^{c} and R^{f} independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR^{c} or —SC(O)R^{c} wherein R^{c} represents a hydrocarbyl.

The term “thioether”, as used herein, is equivalent to an ether, wherein the oxygen is replaced with a sulfur.

The term “urea” is art-recognized and may be represented by the general formula

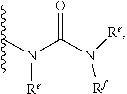

wherein R^{c} and R^{f} independently represent hydrogen or a hydrocarbyl.

The term “modulate” as used herein includes the inhibition or suppression of a function or activity (such as cell proliferation) as well as the enhancement of a function or activity.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula (I). Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula (I) are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula (I) for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula (I) or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of Formula (I)). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs include using ester or phosphoramidate as biologically labile or cleavable (protecting) groups. The prodrugs of this disclosure are metabolized to produce a compound of Formula (I). The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

Unless otherwise indicated in the tables of compounds herein, the abbreviation RAC or rac indicates a racemic mixture, and DIAST indicates a specific diastereomer. In illustrative embodiments, although a compound may be depicted with [symbol] or [symbol] bonds, such a depiction may be denoting relative stereochemistry based on elution peaks from a chiral separation.

### Additional Embodiments

In certain embodiments, the compound is a compound of one or more of the following embodiments, or a pharmaceutically acceptable salt thereof:

1. A compound of Formula (I):

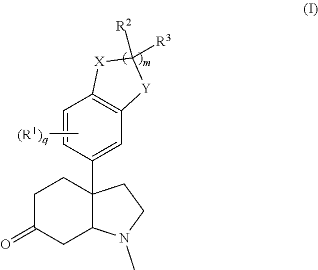

or a pharmaceutically acceptable salt thereof;
wherein
each of X and Y is independently selected from O, NR⁴, and S;
each R¹ is independently deuterium, halo, C₁₋₄ alkyl, C₂₋₄ alkenyl, C₂₋₄ alkynyl, C₃₋₈ cycloalkyl, C₃₋₈ cycloalkenyl, 3 to 8 membered heterocycloalkyl comprising one or more N, O or S heteroatoms, C₅₋₆ aryl, 5-6 membered heteroaryl comprising one or more N, O or S heteroatoms, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocycloalkyl, aryl, and heteroaryl is optionally substituted by halo, C₁₋₄ alkyl, C₁₋₄ alkanol, C₅₋₆ aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H or C₁₋₄ alkyl; wherein each hydrogen atom in alkyl is optionally substituted by halo, deuterium, C₃₋₈ cycloalkyl, or OR^{a}; or R² and R³ taken together with the atoms to which they are attached combine to form carbonyl, C₃₋₈ cycloalkyl, or 3 to 8 membered heterocyclyl; wherein each hydrogen atom in cycloalkyl and heterocyclyl is optionally substituted by halo or OR^{a};
R⁴ is H or C₁₋₄ alkyl;
m is 1 or 2;
q is 0, 1, 2, or 3;
each R^{a} and R^{b} is independently H, C₁₋₄ alkyl, C₂₋₄ alkenyl, C₃₋₈ cycloalkyl, 3-8 membered heterocycloalkyl comprising one or more N, O or S heteroatoms, C₅₋₆ aryl, or 5-6 membered heteroaryl comprising one or more N, O or S heteroatoms; or if an instance of R¹ is —NR^{a}R^{b}, then R^{a} and R^{b} may combine with the nitrogen atom to which they are attached to form 3-8 membered heterocycloalkyl comprising one or more N, O or S heteroatoms or 5-6 membered heteroaryl comprising one or more N, O or S heteroatoms, wherein each hydrogen atom in alkyl, alkenyl, cycloalkyl, heterocycloalkyl, aryl, or heteroaryl is optionally substituted by halo, hydroxy, C₁₋₄ alkyl, C₁₋₄ alkanol, C₅₋₆ aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, C₁₋₄ alkyl, C₂₋₄ alkenyl, C₃₋₈ cycloalkyl, 3-8 membered heterocyclyl comprising one or more N, O or S heteroatoms, C₅₋₆ aryl, or 5-6 membered heteroaryl comprising one or more N, O or S heteroatoms.

2. The compound of embodiment 1, wherein the compound is of Formula (IA):

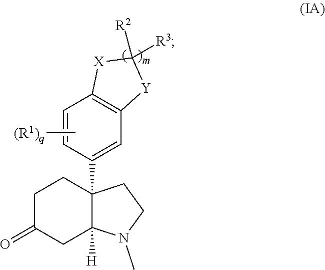

or a pharmaceutically acceptable salt thereof.

3. The compound of embodiment 1, wherein the compound is of Formula (II):

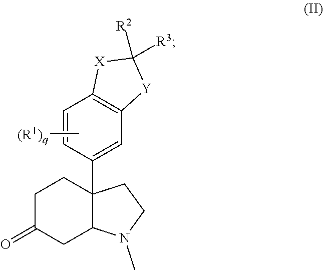

or a pharmaceutically acceptable salt thereof.

4. The compound of embodiment 1, wherein the compound is of Formula (IIA):

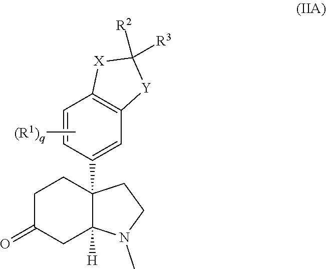

or a pharmaceutically acceptable salt thereof.

5. The compound of any one of embodiments 1-4, wherein q is 1, 2, or 3.

6. The compound of any one of embodiments 1-4, wherein q is 1.

7. The compound of embodiment 1, wherein the compound is of Formula (I-1):

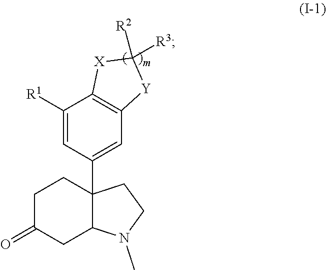

or a pharmaceutically acceptable salt thereof.

8. The compound of embodiment 1, wherein the compound is of Formula (IA-1):

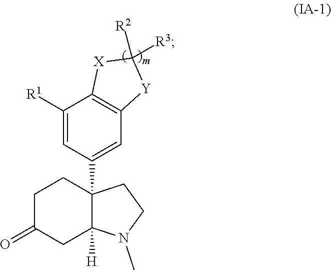

or a pharmaceutically acceptable salt thereof.

9. The compound of embodiment 1, wherein the compound is of Formula (II-1):

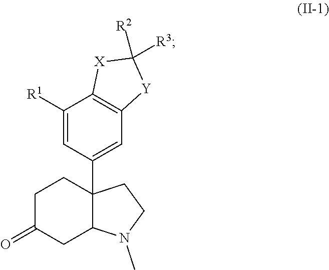

or a pharmaceutically acceptable salt thereof.

10. The compound of embodiment 1, wherein the compound is of Formula (IIA-1):

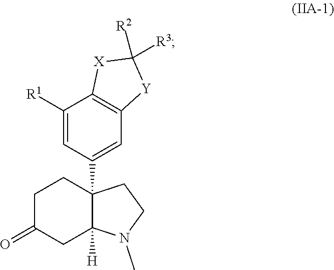

or a pharmaceutically acceptable salt thereof.

11. The compound of any one of embodiments 1-10, wherein each of R² and R³ is H.

12. The compound of any one of embodiments 1-10, wherein one of R² and R³ is halo or alkyl.

13. The compound of any one of embodiments 1-10, wherein one of R² and R³ is fluoro, chloro, bromo, or iodo.

14. The compound of any one of embodiments 1-10, wherein one of R² and R³ is C₁-C₆ alkyl.

15. The compound of any one of embodiments 1-10, wherein one of R² and R³ is methyl.

16. The compound of any one of embodiments 1-10, wherein each of R² and R³ is independently halo or alkyl.

17. The compound of any one of embodiments 1-10, wherein each of R² and R³ is independently selected from fluoro, chloro, bromo, and iodo.

18. The compound of any one of embodiments 1-10, wherein each of R² and R³ is independently C₁-C₆ alkyl.

19. The compound of any one of embodiments 1-10, wherein each of R² and R³ is methyl.

20. The compound of any one of embodiments 1-10, wherein R² and R³ taken together with the carbon to which they are attached combine to form carbonyl, cycloalkyl, or heterocyclyl.

21. The compound of any one of embodiments 1-10, wherein, in one instance, R² and R³ taken together with the carbon to which they are attached combine to form C₃-C₆ cycloalkyl.

22. The compound of any one of embodiments 1-10, wherein, in one instance, R² and R³ taken together with the carbon to which they are attached combine to form C₃-C₄ cycloalkyl.

23. The compound of any one of embodiments 1-10, wherein, in one instance, R² and R³ taken together with the carbon to which they are attached combine to form 3- to 6-membered heterocyclyl.

24. The compound of any one of embodiments 1-10, wherein, in one instance, R² and R³ taken together with the carbon to which they are attached combine to form carbonyl.

25. The compound of embodiment 1, wherein the compound is of Formula (III):

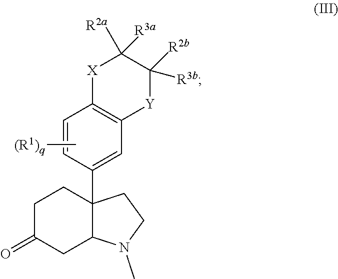

or a pharmaceutically acceptable salt thereof; wherein
each of R^{2a}, R^{3a}, R^{2b} and R^{3b} is independently H or alkyl; wherein each hydrogen atom in alkyl is optionally substituted by halo, deuterium, cycloalkyl, or OR^{a}; or R^{2a} and R^{3a} or R^{2b} and R^{3b} taken together with the atoms to which they are attached combine to form carbonyl, cycloalkyl, or heterocyclyl; wherein each hydrogen atom in cycloalkyl and heterocyclyl is optionally substituted by halo or OR^{a}.

26. The compound of embodiment 25, wherein the compound is of Formula (IIIA):

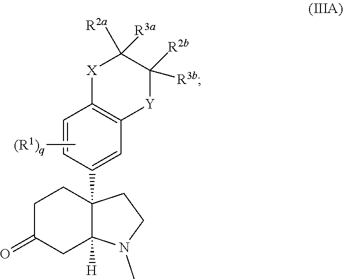

or a pharmaceutically acceptable salt thereof.

27. The compound of embodiment 25 or 26, wherein q is 1, 2, or 3.

28. The compound of embodiment 25 or 26, wherein q is 1.

29. The compound of embodiment 25, wherein the compound is of Formula (III-1):

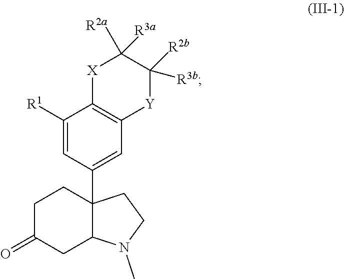

or a pharmaceutically acceptable salt thereof.

30. The compound of embodiment 25, wherein the compound is of Formula (IIIA-1):

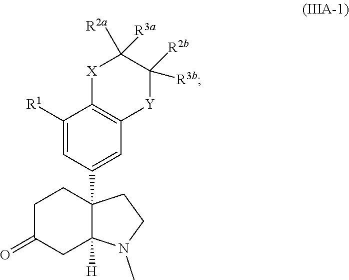

or a pharmaceutically acceptable salt thereof.

31. The compound of any one of embodiments 25-30, wherein each of R^{2a}, R^{3a}, R^{2b} and R^{3b} is H.

32. The compound of any one of embodiments 25-30, wherein one of R^{2a}, R^{3a}, R^{2b} and R^{3b} is halo or alkyl.

33. The compound of any one of embodiments 25-30, wherein one of R^{2a}, R^{3a}, R^{2b} and R^{3b} is fluoro, chloro, bromo, or iodo.

34. The compound of any one of embodiments 25-30, wherein one of R^{2a}, R^{3a}, R^{2b} and R^{3b} is C₁-C₆ alkyl.

35. The compound of any one of embodiments 25-30, wherein one of R^{2a}, R^{3a}, R^{2b} and R^{3b} is methyl.

36. The compound of any one of embodiments 25-30, wherein two of R^{2a}, R^{3a}, R^{2b} and R^{3b} are halo or alkyl.

37. The compound of any one of embodiments 25-30, wherein two of R^{2a}, R^{3a}, R^{2b} and R^{3b} are independently fluoro, chloro, bromo, or iodo.

38. The compound of any one of embodiments 25-30, wherein two of R^{2a}, R^{3a}, R^{2b} and R^{3b} are independently C₁-C₆ alkyl.

39. The compound of any one of embodiments 25-30, wherein two of R^{2a}, R^{3a}, R^{2b} and R^{3b} are methyl.

40. The compound of any one of embodiments 25-39, wherein R^{2a} and R^{3a} or R^{2b} and R^{3b} taken together with the carbon to which they are attached combine to form carbonyl, cycloalkyl, or heterocyclyl.

41. The compound of any one of embodiments 25-39, wherein R^{2a} and R^{3a} or R^{2b} and R^{3b} taken together with the carbon to which they are attached combine to form C₃-C₆ cycloalkyl.

42. The compound of any one of embodiments 25-39, wherein R^{2a} and R^{3a} or R^{2b} and R^{3b} taken together with the carbon to which they are attached combine to form C₃-C₄ cycloalkyl.

43. The compound of any one of embodiments 25-39, wherein R^{2a} and R^{3a} or R^{2b} and R^{3b} taken together with the carbon to which they are attached combine to form 3- to 6-membered heterocyclyl.

44. The compound of any one of embodiments 1-43, wherein each R¹ is independently halo, alkyl, cyano, or nitro.

45. The compound of any one of embodiments 1-43, wherein each R¹ is independently fluoro, chloro, or bromo.

46. The compound of any one of embodiments 1-43, wherein each R¹ is independently C₁-C₆ alkyl.

47. The compound of any one of embodiments 1-43, wherein each R¹ is methyl.

48. The compound of any one of embodiments 1-43, wherein each R¹ is cyano.

49. The compound of any one of embodiments 1-43, wherein each R¹ is nitro.

50. The compound of any one of embodiments 1-49, wherein at least one of X and Y is O.

51. The compound of any one of embodiments 1-50, wherein X is O.

52. The compound of any one of embodiments 1-51, wherein Y is O.

53. The compound of any one of embodiments 1-50, wherein at least one of X and Y is S.

54. The compound of any one of embodiments 1-50, 52, and 53, wherein X is S.

55. The compound of any one of embodiments 1-51, 53, and 54, wherein Y is S.

56. The compound of any one of embodiments 1-50, 52, 53, and 55, wherein X is NR⁴.

57. The compound of any one of embodiments 1-56, wherein R⁴ is C₁-C₆ alkyl.

58. The compound of any one of embodiments 1-4, 11-26, 31-43, and 50-57, wherein q is 0.

59. The compound of embodiment 1, wherein the compound is of Formula (IIA-1) or Formula (III-1):

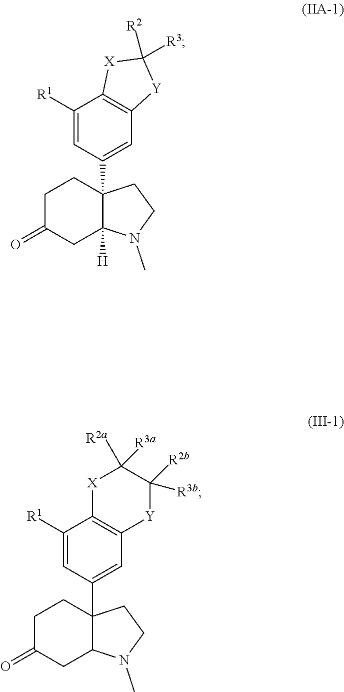

or a pharmaceutically acceptable salt thereof; wherein:
each of X and Y is independently selected from O, NR⁴ and S;
each R¹ is independently hydrogen, deuterium, halo, alkyl, alkenyl, alkynyl, cycloalkyl, cycloalkenyl, heterocycloalkyl, aryl, heteroaryl, —OR^{a}, —NR^{a}R^{b}, —CHO, —C(O)R^{a}, —CO₂R^{a}, C(O)NR^{a}R^{b}, —CN, or nitro; wherein each hydrogen atom in alkyl, alkenyl, alkynyl, cycloalkyl, cycloalkenyl, heterocycloalkyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O)NR^{a}R^{b}, —CN, or nitro;
each of R² and R³ is independently H or alkyl; wherein each hydrogen atom in alkyl is optionally substituted by halo, deuterium, cycloalkyl, or OR^{a}; or R² and R³ taken together with the atoms to which they are attached combine to form carbonyl, cycloalkyl, or heterocyclyl; wherein each hydrogen atom in cycloalkyl and heterocyclyl is optionally substituted by halo or OR^{a};
each of R^{2a}, R^{3a}, R^{2b} and R^{3b} is independently H or alkyl; wherein each hydrogen atom in alkyl is optionally substituted by halo, deuterium, cycloalkyl, or OR^{a}; or R^{2a} and R^{3a} or R^{2b} and R^{3b} taken together with the atoms to which they are attached combine to form carbonyl, cycloalkyl, or heterocyclyl; wherein each hydrogen atom in cycloalkyl and heterocyclyl is optionally substituted by halo or OR^{a};
each R^{a} and R^{b} is independently H, alkyl, alkenyl, cycloalkyl, heterocycloalkyl, aryl, or heteroaryl; or if an instance of R¹ is —NR^{a}R^{b}, then R^{a} and R^{b} may combine with the nitrogen atom to which they are attached to form heterocycloalkyl or heteroaryl, wherein each hydrogen atom in alkyl, alkenyl, cycloalkyl, heterocycloalkyl, aryl, or heteroaryl is optionally substituted by halo, hydroxy, alkyl, alkanol, aryl, —OR^{c}, —NR^{c}R^{d}, —CHO, —C(O)R^{c}, —CO₂R^{c}, —C(O)NR^{c}R^{d}, —CN, or nitro.

60. The compound of embodiment 59, wherein each R¹ is independently hydrogen, deuterium, halo, C₁-C₄ alkyl, nitro, or CN.

61. The compound of embodiment 59, wherein each R¹ is hydrogen.

62. The compound of embodiment 59, wherein each R¹ is methyl.

63. The compound of embodiment 59, wherein each R¹ is fluoro, chloro, bromo or iodo.

64. The compound of embodiment 59, wherein each R¹ is fluoro.

65. The compound of embodiment 59, wherein each R¹ is chloro.

66. The compound of embodiment 59, wherein each R¹ is bromo.

67. The compound of embodiment 59, wherein each R¹ is iodo.

68. The compound of embodiment 59, wherein each R¹ is nitro.

69. The compound of embodiment 59, wherein each R¹ is CN.

70. The compound of any one of embodiments 59-69, wherein each of R² and R³ is independently H or C₁-C₄ alkyl; wherein each hydrogen atom in alkyl is optionally substituted by halo, deuterium, C₃-C₆ cycloalkyl, or OR^{a}; or R² and R³ taken together with the atoms to which they are attached combine to form carbonyl, C₃-C₆ cycloalkyl, or 3-6 membered heterocyclyl; wherein each hydrogen atom in cycloalkyl and heterocyclyl is optionally substituted by halo or OR^{a};
each of R^{2a}, R^{3a}, R^{2b} and R^{3b} is independently H or C₁-C₄ alkyl; wherein each hydrogen atom in alkyl is optionally substituted by halo, deuterium, C₃-C₆ cycloalkyl, or OR^{a}; or R^{2a} and R^{3a} or R^{2b} and R^{3b} taken together with the atoms to which they are attached combine to form carbonyl, C₃-C₆ cycloalkyl, or 3-6 membered heterocyclyl; wherein each hydrogen atom in cycloalkyl and heterocyclyl is optionally substituted by halo or OR^{a}; and
each R^{a} is independently H, or C₁-C₄ alkyl; wherein each hydrogen atom in alkyl is optionally substituted by halo, hydroxy, —CN, or nitro.

71. The compound of embodiment 70, wherein each of R² and R³ is independently H; and
each of R^{2a}, R^{3a}, R^{2b} and R^{3b} is H.

72. The compound of embodiment 70, wherein the compound is a compound of Formula (IIA-1).

73. The compound of embodiment 72, wherein each of R² and R³ is independently halo or H.

74. The compound of embodiment 72, wherein each of R² and R³ is independently fluoro or H.

75. The compound of embodiment 72, wherein each of R² and R³ is H.

76. The compound of embodiment 72, wherein each of R² and R³ is fluoro.

77. The compound of embodiment 72, wherein R² and R³ taken together with the carbon atom to which they are attached combine to form carbonyl.

78. The compound of embodiment 72, wherein each of R² and R³ is independently H or C₁-C₄ alkyl.

79. The compound of embodiment 72, wherein each of R² and R³ is independently H or methyl.

80. The compound of embodiment 72, wherein R² is methyl; and R³ is H or methyl.

81. The compound of embodiment 72, wherein each of R² and R³ is independently H or methyl.

82. The compound of embodiment 72, wherein R² and R³ taken together with the atoms to which they are attached combine to form C₃-C₆ cycloalkyl or 3-6 membered heterocyclyl.

83. The compound of embodiment 82, wherein R² and R³ taken together with the atoms to which they are attached combine to form cyclopropyl.

84. The compound of embodiment 82, wherein R² and R³ taken together with the atoms to which they are attached combine to form cyclobutyl.

85. The compound of embodiment 82, wherein R² and R³ taken together with the atoms to which they are attached combine to form oxetanyl.

86. The compound of embodiment 70, wherein the compound is a compound of Formula (III-1).

87. The compound of embodiment 86, wherein each of R^{2a}, R^{3a}, R^{2b} and R^{3b} is independently H, halo or C₁-C₄ alkyl; wherein each hydrogen atom in alkyl is optionally substituted by halo.

88. The compound of embodiment 86, wherein each of R^{2a}, R^{3a}, R^{2b} and R^{3b} is independently H, fluoro or methyl.

89. The compound of any one of embodiments 59-88, wherein X is O.

90. The compound of any one of embodiments 59-88, wherein X is NR⁴.

91. The compound of any one of embodiments 59-88, wherein X is S.

92. The compound of any one of embodiments 59-91, wherein Y is O.

93. The compound of any one of embodiments 59-91, wherein Y is NR⁴.

94. The compound of any one of embodiments 59-91, wherein Y is S.

95. The compound of any one of embodiments 59-94, wherein R⁴ is H.

96. The compound of any one of embodiments 59-94, wherein R⁴ is methyl.

97. The compound of any one of embodiments 59-95, wherein R^{a} is methyl.

98. The compound of any one of embodiments 59-95, wherein R^{a} is H.

99. The compound of any one of embodiments 59-98, wherein R^{b} is H.

100. The compound of any one of embodiments 59-98, wherein R^{b} is methyl.

101. The compound of any one of embodiments 59-100 wherein R^{c} is H.

102. The compound of any one of embodiments 59-100, wherein R^{c} is methyl.

103. The compound of any one of embodiments 59-102 wherein R^{d} is H.

104. The compound of any one of embodiments 59-102, wherein R^{d} is methyl.

105. The compound of embodiment 1, selected from:

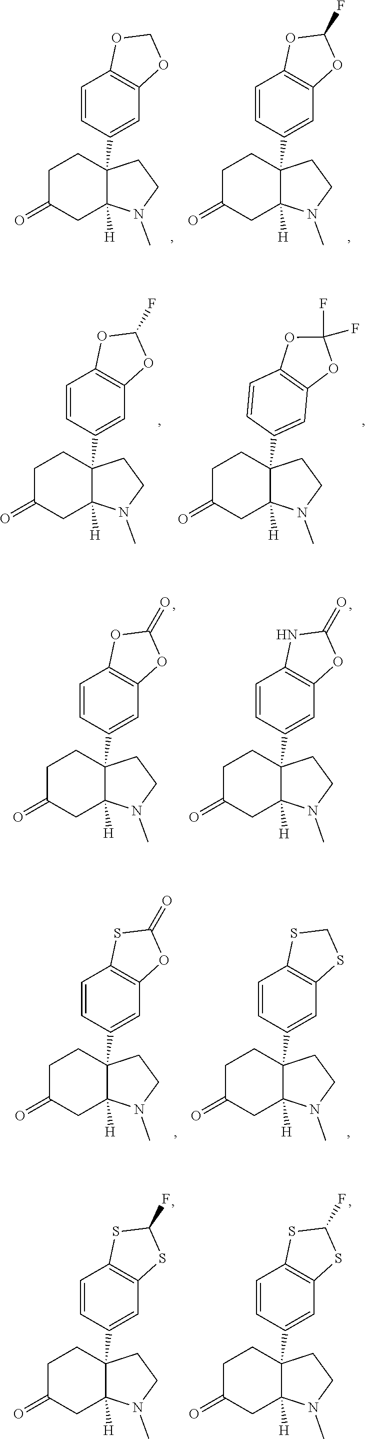

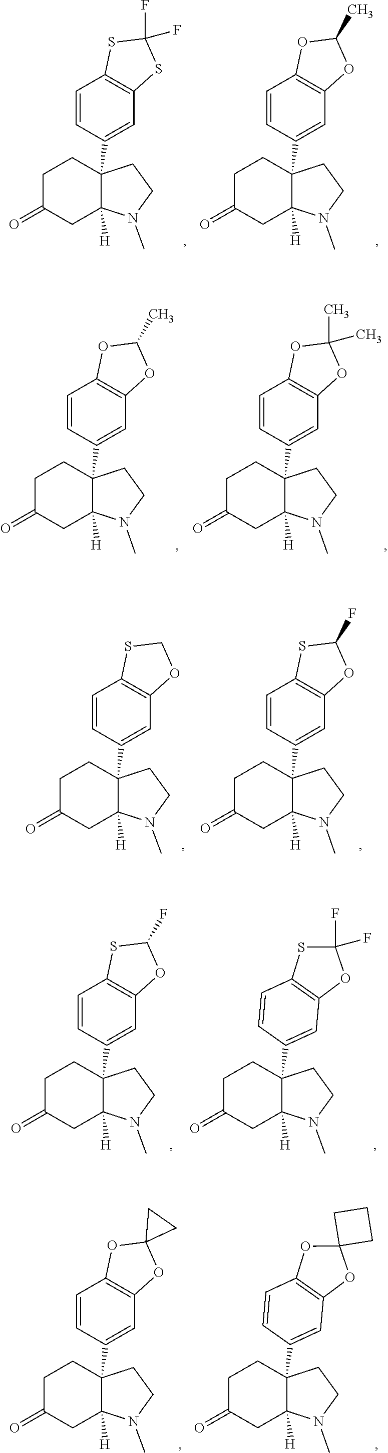

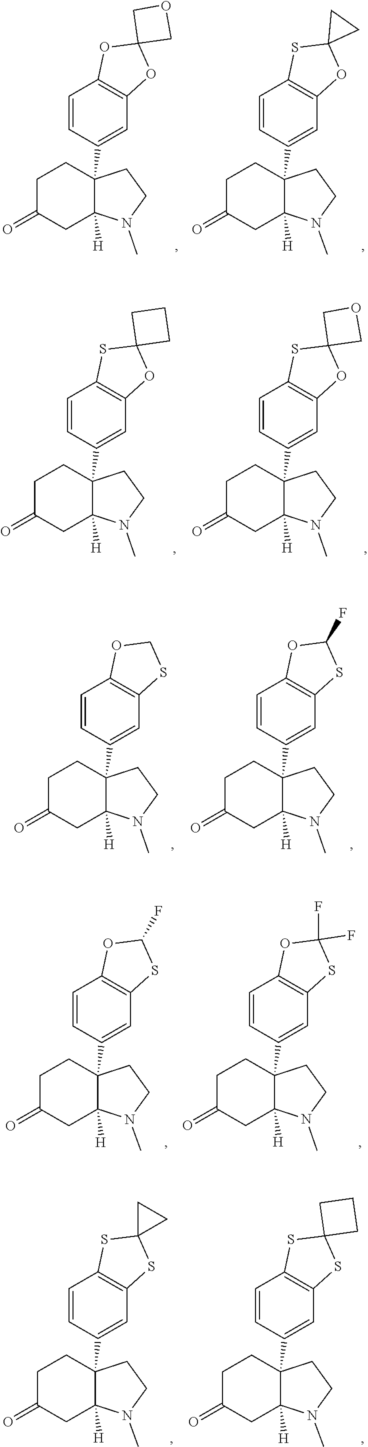

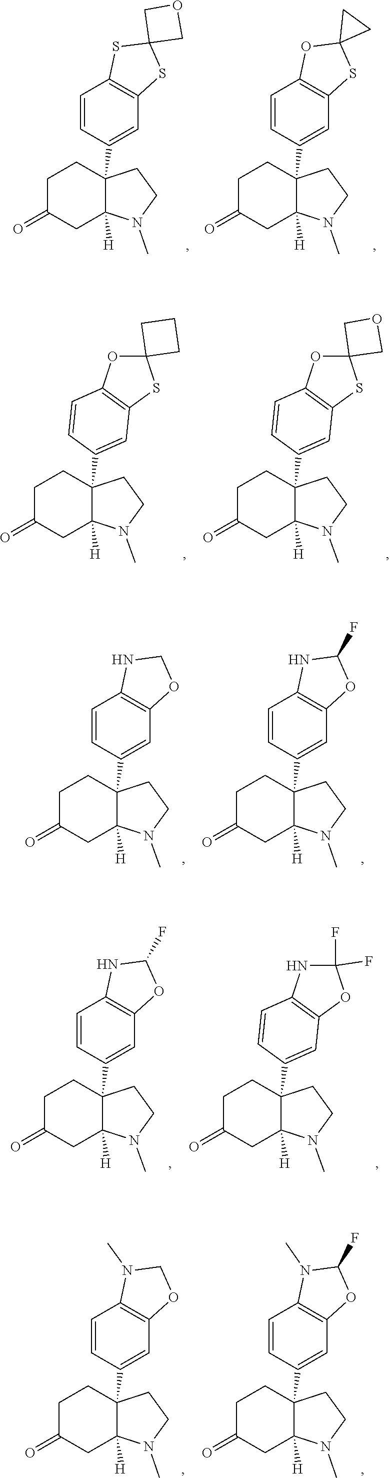

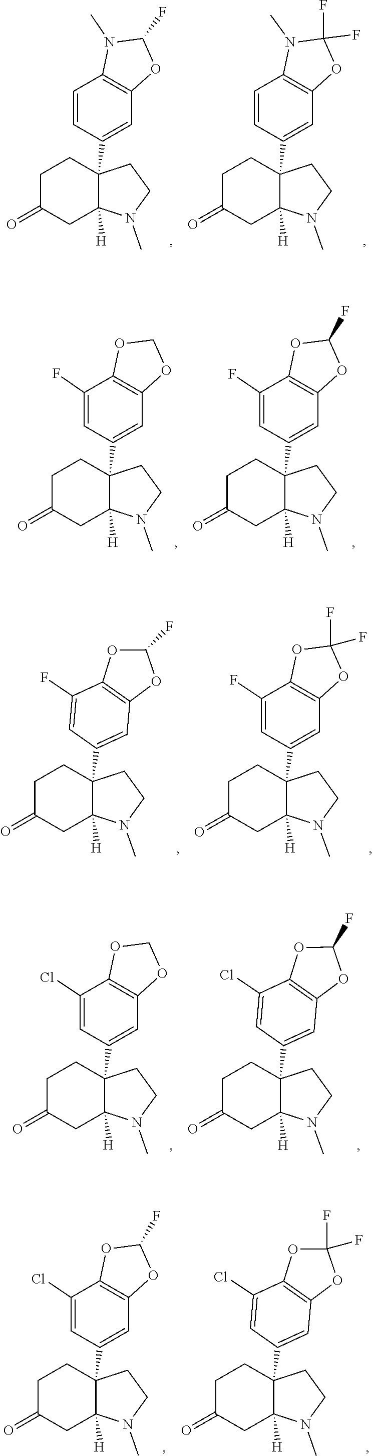

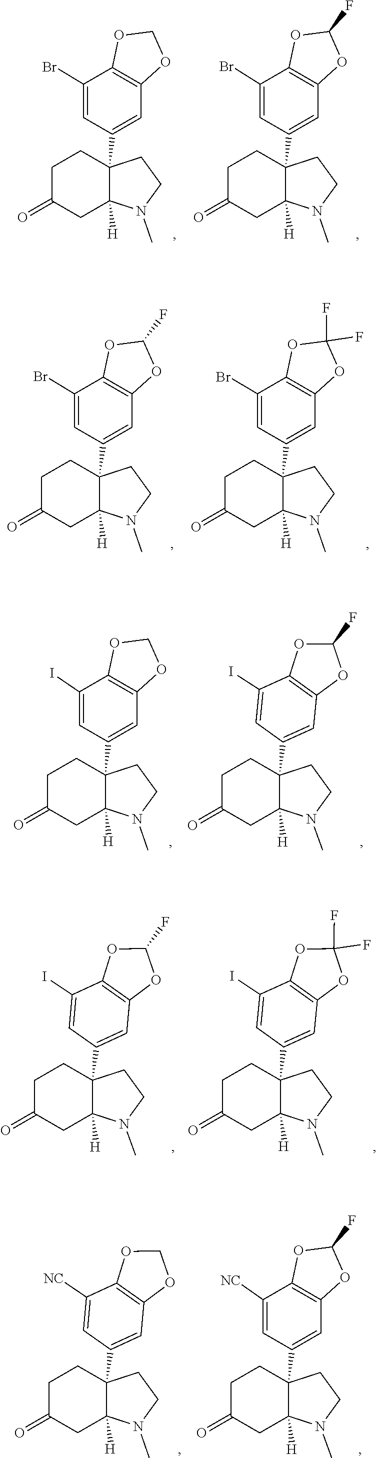

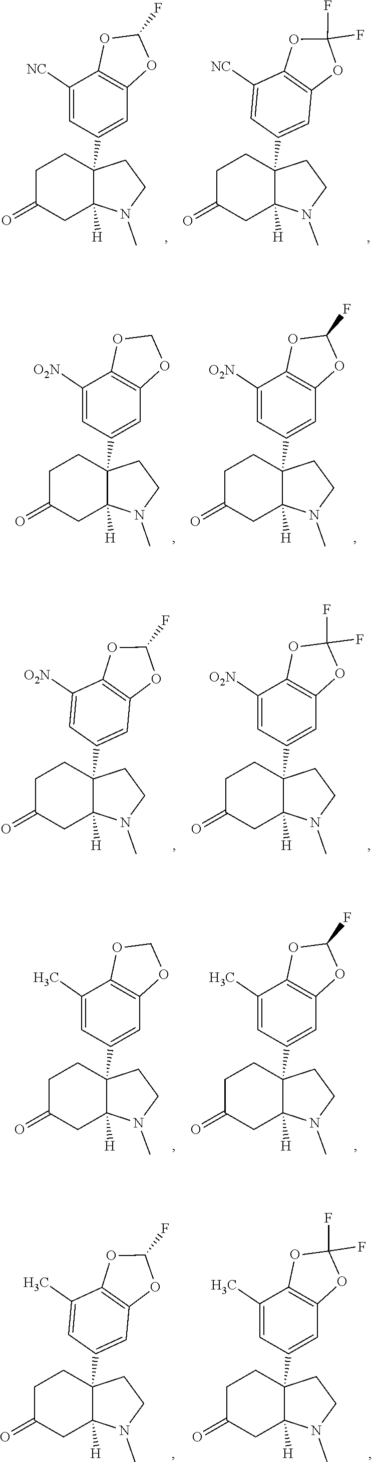

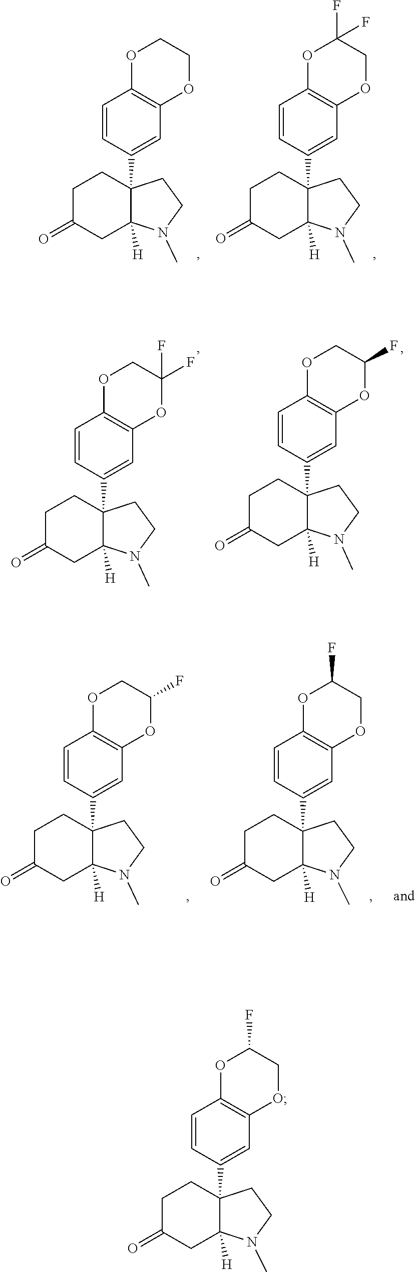

or a pharmaceutically acceptable salt thereof.

106. The compound of any one of embodiments 2, 4-6, 8, 10-24, 26-28, and 30-105, wherein the compound has the absolute stereochemistry shown.

107. A pharmaceutical composition, comprising a compound of any one of embodiments 1-106; and a pharmaceutically acceptable excipient.

108. A method of treating a mental health disorder, comprising administering to a mammal in need thereof an effective amount of a compound according to any one of embodiments 1-106 or a pharmaceutically acceptable salt thereof.

109. The method of embodiment 108, wherein the mental health disorder is anxiety, stress, or depression.

110. The method of embodiment 108, wherein the mental health disorder is anxiety.

111. The method of embodiment 108, wherein the mental health disorder is stress.

112. The method of embodiment 108, wherein the mental health disorder is depression.

113. The method of any one of embodiments 108-112, wherein the mammal is a human.

114. A method of treating an inflammatory condition, comprising administering to a mammal in need thereof an effective amount of a compound according to any one of embodiments 1-106 or a pharmaceutically acceptable salt thereof.

115. The method of embodiment 114, wherein the inflammatory condition is chronic obstructive pulmonary disease (COPD), asthma, or rheumatoid arthritis.

116. The method of embodiment 114, wherein the inflammatory condition is COPD.

117. The method of embodiment 114, wherein the inflammatory condition is asthma.

118. The method of embodiment 114, wherein the inflammatory condition is rheumatoid arthritis.

119. The method of any one of embodiments 114-118, wherein the mammal is a human.

120. The method of any one of embodiments 108-119, wherein the compound is administered parenterally (e.g., intravenously, subcutaneously, or intramuscularly).

121. The method of any one of embodiments 108-119, wherein the compound is administered orally (e.g., in the form of a tablet or capsule).

### EXAMPLES

Certain compounds in the following examples are labeled using the MDL enhanced stereorepresentation. For example, the label “ABS” denotes the absolute stereochemistry at a particular stereocenter. The label “or n” or “orn” where n is an integer (e.g., “orn”), denotes a stereoisomer that has either the stereochemistry as drawn or is the epimer at that particular stereocenter. The label “and n” or “&n,” where n is an integer (e.g., “and 1” or “&1”) represents a mixture of two epimers at the stereocenter, i.e., the structure as drawn and the epimer in which the stereogenic center has the opposite configuration (e.g., a racemic mixture).

LC/MS spectra were obtained using Agilent 1200\G1956A or SHIMADZU LCMS-2020. Standard LC/MS conditions were as follows (running time 1.55 minutes):

Acidic condition: Mobile Phase A: 0.0375% TFA in water (v/v). Mobile Phase B: 0.01875% TFA in acetonitrile (v/v); Column: Kinetex EVO C18 30*2.1 mm, 5 μm.

Basic condition: Mobile Phase A: 0.025% NH₃·H₂O in water (v/v). Mobile Phase B: Acetonitrile; Column: Kinetex EVO C18 2.1×30 mm, 5 μm.

5-95AB_0.8 min

| Instrument | | SHIMADZU LCMS-2020; | | |
|---|---|---|---|---|
| Software | | LabSolution Version 5.97SP1 | | |
| HPLC | Column | Kinetex ® EVO C18 2.1 × 30 mm 5 um | | |
| | Mobile Phase | A: 0.0375% TFA in water (v/v) | | |
| | | B: 0.01875% TFA in Acetonitrile (v/v) | | |
| | | Time(min) | B(%) | Flow(mL/min) |
| | Gradient | 0.00 | 5.0 | 2.0 |
| | | 0.60 | 95.0 | 2.0 |
| | | 0.78 | 95.0 | 2.0 |
| | | 0.79 | 5.0 | 2.0 |
| | | 0.80 | 5.0 | 2.0 |

| | | |
|---|---|---|
| | Column Temp | 50° C. |
| | Detector | PDA (220 nm&254 nm) |
| MS | Ionization source | ESI |
| | Drying Gas | N2 |
| | Drying Gas Flow | 15(L/min) |
| | DL Voltage | 120(v) |
| | Qarray DC Voltage | 20(V) |
| | MS Polarity | Positive |
| | MS Mode | Scan |
| | Mass range | 100-1000 |

Table of Abbreviations

| | |
|---|---|
| Ac | Acetyl |
| Et | Ethyl |
| Me | Methyl |
| Ms | Methanesulfonyl |
| Ts | p-Toluenesulfonyl |
| Tol | toluene |
| HPLC | High Performance Liquid Chromatography |
| DMSO | Dimethyl sulfoxide |
| LC-MS | Liquid chromatography - mass spectrometry |
| NMR | Nuclear magnetic resonance |
| m | multiplet |
| d | Doublet |
| s | singlet |
| t | triplet |
| SFC | Supercritical fluid chromatography |
| IPA | isopropanol |
| ESI | Electrospray ionization |
| EtOAC | Ethyl acetate |
| t-buOK | Potassium tertbutoxide |
| ACN | acetonitrile |
| Ac | acetate |
| MS | Molecular sieves |
| DCM | dichloromethane |
| TMS | trimethylsilyl |

### Example 1: Synthesis of 149 and 150

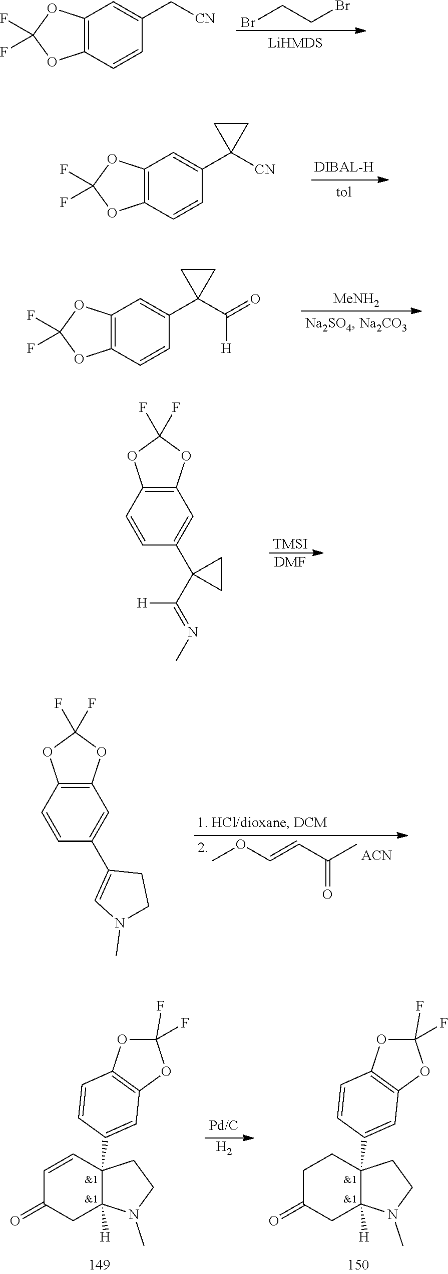

### Step 1: Synthesis of 1-(2,2-difluorobenzo[d][1,3]dioxol-5-yl)cyclopropane-1-carbonitrile

To a solution of 1,2-dibromoethane (19.0 g, 101 mmol, 7.7 mL) in THF (300 mL) was added LiHMDS (1 M, 177.5 mL). The mixture was allowed to stir at −20° C. for 30 min, then 2-(2,2-difluoro-1,3-benzodioxol-5-yl)acetonitrile (10 g, 50.7 mmol) was added. The reaction mixture was allowed to stir and warm from −20° C. to 25° C. over 2 hr. The reaction mixture was quenched by pouring into cold saturated aqueous NH₄CI solution (300 mL). The aqueous solution was extracted with ethyl acetate (300 mL×2). The organic solutions were combined, dried over anhydrous Na₂SO₄, filtered and concentrated under reduced pressure. The residue was purified by column chromatography (SiO₂, Petroleum ether/Ethyl acetate=10/1 to 10/1) to give 1-(2,2-difluorobenzo[d][1,3]dioxol-5-yl)cyclopropane-1-carbonitrile (7.5 g, 50%) as yellow oil. ¹H NMR (400 MHz, CDCl₃) δ 7.09-6.91 (m, 3H), 1.70-1.65 (m, 2H), 1.35-1.26 (m, 2H).

### Step 2: Synthesis of 1-(2,2-difluorobenzo[d][1,3]dioxol-5-yl)cyclopropane-1-carbaldehyde

To a solution of 1-(2,2-difluorobenzo[d][1,3]dioxol-5-yl)cyclopropane-1-carbonitrile (7.5 g, 33.6 mmol) in toluene (70 mL) was added DIBAL-H (1 M, 40.3 mL). The mixture was allowed to stir at 0-25° C. for 8 hr. The reaction mixture was quenched by pouring into cold saturated aqueous 2N HCl (50 mL). The aqueous solution was extracted with ethyl acetate (50 mL×2). The organic solutions were combined, dried over anhydrous Na₂SO₄, filtered and concentrated under reduced pressure. The residue was purified by column chromatography (SiO₂, Petroleum ether/Ethyl acetate=10/1 to 10/1) to give 1-(2,2-difluorobenzo[d][1,3]dioxol-5-yl)cyclopropane-1-carbaldehyde (6.2 g, 85%) as yellow oil. ¹H NMR (400 MHz, CDCl₃) δ 9.33 (s, 1H), 7.64-7.38 (m, 1H), 7.35-7.27 (m, 1H), 7.24 (d, J=1.6 Hz, 1H), 1.88-1.81 (m, 2H), 1.69-1.62 (m, 2H).

### Step 3: Synthesis of (E)-1-(1-(2,2-difluorobenzo[d][1,3]dioxol-5-yl)cyclopropyl)-N-methylmethanimine

To a solution of 1-(2,2-difluorobenzo[d][1,3]dioxol-5-yl)cyclopropane-1-carbaldehyde (6.2 g, 27.4 mmol) in DCM (150 mL) was added methanamine (2M, 137 mL) and Na₂SO₄ (17.5 g, 123 mmol, 12.5 mL). The mixture was allowed to stir at 25° C. for 10 hr. The reaction mixture was quenched by adding it to a cold saturated aqueous H₂O solution (150 mL). The aqueous solution was extracted with ethyl acetate (150 mL×2). The organic solutions were combined, dried over anhydrous Na₂SO₄, filtered and concentrated under reduced pressure to give (E)-1-(1-(2,2-difluorobenzo[d][1,3]dioxol-5-yl)cyclopropyl)-N-methylmethanimine (6.7 g) as yellow oil. ¹H NMR (400 MHz, CDCl₃) δ 9.02 (s, 1H), 6.99-6.86 (m, 3H), 3.16 (d, J=1.2 Hz, 3H), 1.23-1.16 (m, 2H), 1.12-1.05 (m, 2H).

### Step 4: Synthesis of 4-(2,2-difluorobenzo[d][1,3]dioxol-5-vl)-1-methyl-2,3-dihydro-1H-pyrrole

A mixture of (E)-1-(1-(2,2-difluorobenzo[d][1,3]dioxol-5-yl)cyclopropyl)-N-methylmethanimine (500 mg, 2.09 mmol) and TMSI (418 mg, 2.09 mmol, 284 uL) in DMF (4 mL) was degassed and purged with N₂ 3 times, and then the reaction mixture was allowed to stir at 60° C. for 1 hr under an atmosphere of N₂. The reaction mixture was quenched by adding it to water (10 mL). The aqueous solution was extracted with ethyl acetate (10 mL×2). The organic solutions were combined, dried over anhydrous Na₂SO₄, filtered and concentrated under reduced pressure to give 4-(2,2-difluorobenzo[d][1,3]dioxol-5-yl)-1-methyl-2,3-dihydro-1H-pyrrole (1.5 g, 60%) as a yellow oil. ¹H NMR (400 MHz, CDCl₃) δ 6.95-6.89 (m, 2H), 6.85-6.80 (m, 1H), 6.36 (s, 1H), 2.68 (s, 3H).

### Step 5: Synthesis of 149

A mixture of 4-(2,2-difluorobenzo[d][1,3]dioxol-5-yl)-1-methyl-2,3-dihydro-1H-pyrrole (1.2 g, 5.02 mmol), (E)-4-methoxybut-3-en-2-one (753 mg, 7.52 mmol, 755 uL), HCl/dioxane (4 M, 123 mL), DCM (5 mL) and ACN (5 mL) was degassed and purged with N₂ 3 times, and then the reaction mixture was allowed to stir at 90° C. for 8 hr under an atmosphere of N₂. The reaction mixture was filtered and the filtrate was concentrated under reduced pressure. The residue was purified by prep-HPLC (column: Welch Xtimate C18 150*25 mm*5 um; mobile phase: [water (HCl)-ACN]; B %: 7%-37%, 8 min) to give 149 (1 g, 46%) as a white solid. LC-MS (ESI⁺) m/z 308.0 (M+H)⁺. ¹H NMR (400 MHz, DMSO-d₆) δ 10.69-10.47 (m, 1H), 7.72 (s, 1H), 7.45 (d, J=8.4 Hz, 1H), 7.28 (dd, J=1.6, 8.5 Hz, 1H), 7.04 (d, J=9.2 Hz, 1H), 6.27 (d, J=10.4 Hz, 1H), 4.06 (s, 1H), 3.57 (dd, J=7.6, 11.2 Hz, 1H), 3.46-3.39 (m, 1H), 2.96 (d, J=4.4 Hz, 3H), 2.84-2.71 (m, 2H), 2.49-2.41 (m, 2H).

### Step 6: Synthesis of 150

To a solution of 149 (140 mg, 455 umol) in EtOAc (3 mL) was added Pd/C (15 mg, 455 umol, 10% purity). The reaction mixture was allowed to stir at 25° C. for 2 hr under an atmosphere of H₂. The reaction mixture was filtered and the filtrate was concentrated under reduced. The residue was purified by prep-HPLC (column: Welch Xtimate C18 150*25 mm*5 um; mobile phase: [water (ammonia hydroxide v/v)-ACN]; B %: 39%-69%, 2 min) to give 150 (80 mg, 54%) as white oil. LC-MS (ESI+) m/z 303.1 (M+H)+. ¹H NMR (400 MHz, CDCl3) δ 7.15-7.08 (m, 2H), 7.06-7.02 (m, 1H), 3.19 (d, J=2.4 Hz, 1H), 2.96 (s, 1H), 2.61 (d, J=2.4 Hz, 2H), 2.52-2.42 (m, 1H), 2.35 (s, 3H), 2.30-2.22 (m, 1H), 2.20-2.09 (m, 4H), 1.71-1.41 (m, 1H).

### Example 2: Prep-HPLC Separation of 150 to Give 232 and 233

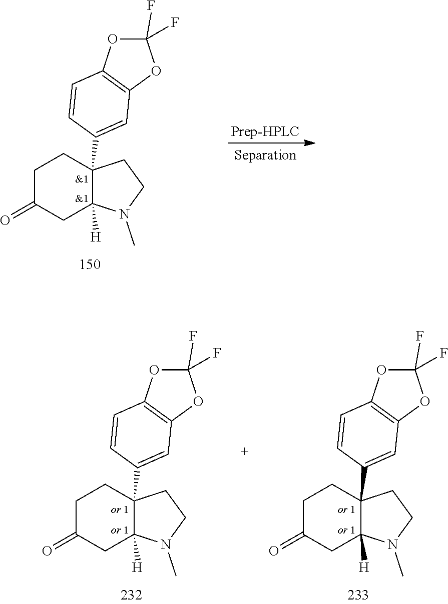 [SMILES: CN1CC[C@]2(c3ccc4c(c3)OC(F)(F)O4)CCC(=O)C[C@H]12 ; "Prep-HPLC" ; CN1CC[C@]2(c3ccc4c(c3)OC(F)(F)O4)CCC(=O)C[C@H]12 ; CN1CC[C@@]2(c3ccc4c(c3)OC(F)(F)O4)CCC(=O)C[C@@H]12]

The enantiomers of 150 were separated by prep-HPLC (column: DAICEL CHIRALPAK AD (250 mm*30 mm, 10 um); mobile phase: [Neu-MeOH]; B %: 25%-25%, C6.0; 54 min) to 232 (34 mg, 42%) as yellow oil and 233 (35 mg, 53%) as white oil.

232: LC-MS (ESI⁺) m/z 310.1 (M+H)⁺. ¹H NMR (400 MHz, CDCl₃) δ 7.18-7.08 (m, 2H), 7.07-7.02 (m, 1H), 3.23-3.11 (m, 1H), 2.93 (s, 1H), 2.67-2.54 (m, 2H), 2.53-2.42 (m, 1H), 2.40-2.29 (m, 4H), 2.26-2.22 (m, 1H), 2.21-2.16 (m, 1H), 2.16-2.07 (m, 3H).

233: LC-MS (ESI⁺) m/z 310.1 (M+H)⁺. ¹H NMR (400 MHz, CDCl₃) δ 7.15-7.08 (m, 2H), 7.06-7.02 (m, 1H), 3.21-3.11 (m, 1H), 2.93 (t, J=3.2 Hz, 1H), 2.60 (dd, J=3.6, 6.1 Hz, 2H), 2.52-2.43 (m, 1H), 2.39-2.29 (m, 4H), 2.28-2.22 (m, 1H), 2.21-2.16 (m, 1H), 2.16-2.06 (m, 3H).

### Example A1: SERT Inhibition Assay

SERT inhibition can be measured using a Neruotransmitter Transportation Fluorescence assay. Briefly, stable 5HTT cells were prepared in a 384 microwell plate. Compounds were prepared by in assay buffer (20 mM HEPES, 0.1% BSA). The compounds were added to the plated cells and incubated for 30 minutes at 37° C. 25 μL of dye solution (Molecular Devices Neurotransmitter Transporter Uptake Assay Kit) is added per well and incubated for 30 minutes at 37° C. The plates are then read on a plate reader.

The results can be provided as follows: A: IC50</=50 nM or lower; B: 50 nM<IC50</=100 nM; C: 100 nM<IC50</=500 nM; D: 500 nM<IC50</=1.5 micromolar; E: IC50>1.5 micromolar.

| Patent | SERT | | |
|---|---|---|---|
| Com- | Assay | IC₅₀ ratio to | |
| pound | Potency | (−) | |
| No. | Range | mesembrine | Structure |
| 001 | A | 1.00 | 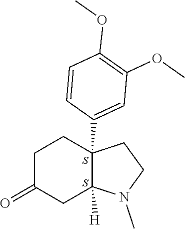 |
| 002 | B | ≥10 | 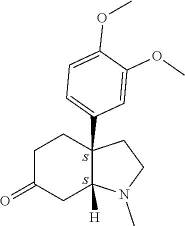 |
| 016 | C | ≥10 | 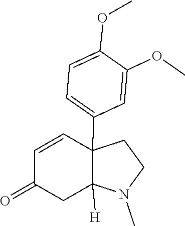 [SMILES: COc1ccc(C23C=CC(=O)CC2N(C)CC3)cc1OC] |
| 233 | D | ≥100 | 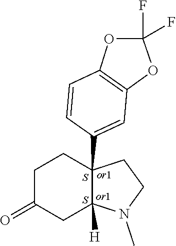 |
| 232 | A | <5 | 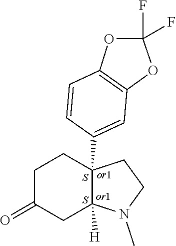 |

### Example A2: PDE4 Inhibition Assay

### Recombinant PDE Assay

Recombinant PDE assay inhibition can be measured according to the BPSBioscience PDE4 assay kit, as described below.

### Step 1:

1) Dilute 20 μM FAM-Cyclic-3′, 5′-AMP stock 100-fold with PDE buffer to make a 200 nM solution. Make only sufficient quantity needed for the assay; store remaining 20 μM stock solution in aliquots at −20° C.
2) Add 25 μl of FAM-Cyclic-3′,5′-AMP (200 nM) to each well designated “Positive Control”, “Test Inhibitor”, and “Substrate Control”.
3) Add 20 μl of PDE assay buffer to each well designated “Substrate Control” and 45 μL of PDE assay buffer to each well designated “Blank”.
4) Add 5 μL of inhibitor solution to each well designated “Test Inhibitor”. For the wells labeled “Positive Control”, “Substrate Control” and “Blank”, add 5 μl of the same solution without inhibitor (inhibitor buffer).
5) Thaw PDE on ice. Upon first thaw, briefly spin tube containing enzyme to recover the full contents of the tube.
6) Dilute PDE4 in PDE buffer to 7.5 pg/μl (0.15 ng/reaction)*. Initiate reaction by adding 20 μL of PDE4 (7.5 pg/μl) to the wells designated “Positive Control” and “Test Inhibitor.”
7) Incubate at room temperature for 1 hour.

### Step 2:

1) Mix binding agent thoroughly and dilute binding agent 1:100 with binding agent diluent.
2) Add 100 μl diluted binding agent to each microwell. Incubate at room temperature for 1 hour with slow shaking.
3) Read the fluorescent polarization of the sample in a microtiter-plate reader equipped for the measurement of fluorescence polarization, capable of excitation at wavelengths ranging from 485±5 nm and detection of emitted light ranging from 528±10 nm. Blank value is subtracted from all other values.

### Cell-Based Assay

Cells can be dispensed at a density of 1000 cells/well in black, clear bottom, tissue culture treated, 1536 well plates (Kalypsys, San Diego, CA) in 3 μl assay medium containing DMEM, 2% FBS, 50 units/mL penicillin and 50 μg/mL streptomycin and can be incubated 24 hr at 37° C. with 5% CO₂ prior to compound screening. 3 μl/well of 1×membrane potential dye was added and incubated for 1 hr at the room temperature. The library compounds in DMSO solution or the positive control, RO 20-1724, can be added at 23 nL/well with a Pintool Station (Kalypsys, San Diego, CA). After a 30-minute incubation with compounds at the room temperature, the assay plate can be measured in an Envision fluorescence plate reader (PerkinElmer, Woburn, MA) in bottom reading mode with an excitation of 535 (±20) nm and emission of 590 (±20) nm. A flying reagent dispensing (FRD) workstation (Aurora Discovery, San Diego, CA) can be used to dispense cells and reagents to 1536-well plates. The Kalypsys Pintool Station can be used to transfer 23 nL compounds in DMSO solution to the 1536-well assay plate. The final DMSO concentration in the assay plates can be under 0.5%.

We claim:

1.-37. (canceled)

38. A compound of Formula (I): 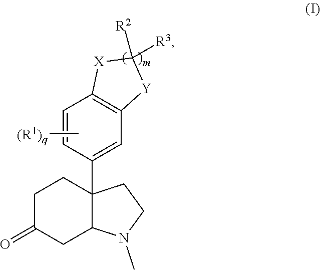
or a pharmaceutically acceptable salt thereof;
wherein
each of X and Y is independently selected from O, NR⁴, and S;
each R¹ is independently deuterium, halo, C₁₋₄ alkyl, C₁₋₄ alkenyl, C₁₋₄ alkynyl, C₃₋₈ cycloalkyl, C₃₋₈ cycloalkenyl, 3- to 8-membered heterocycloalkyl comprising one or more N, O or S heteroatoms, C₅₋₆ aryl, 5- to 6-membered heteroaryl comprising one or more N, O or S heteroatoms, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocycloalkyl, aryl, and heteroaryl is optionally substituted by halo, C₁₋₄ alkyl, C₁₋₄ alkanol, C₅₋₆ aryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b};
each of R² and R³ is independently H, halo, or C₁₋₄ alkyl; wherein each hydrogen atom in alkyl is optionally substituted by halo, deuterium, C₃₋₈ cycloalkyl, or OR^{a}; or R² and R³ taken together with the atoms to which they are attached combine to form carbonyl, C₃₋₈ cycloalkyl, or 3- to 8-membered heterocyclyl comprising one or more N, O or S heteroatoms; wherein each hydrogen atom in cycloalkyl and heterocyclyl is optionally substituted by halo or OR^{a};
R⁴ is H or C₁₋₄ alkyl;
m is 1 or 2;
q is 0, 1, 2, or 3;
each R^{a} and R^{b} is independently H, C₁₋₄ alkyl, C₁₋₄ alkenyl, C₃₋₈ cycloalkyl, 3- to 8-membered heterocycloalkyl comprising one or more N, O or S heteroatoms, C₅₋₆ aryl, or 5- to 6-membered heteroaryl comprising one or more N, O or S heteroatoms; or if an instance of R¹ is —NR^{a}R^{b}, then R^{a} and R^{b} may combine with the nitrogen atom to which they are attached to form 3- to 8-membered heterocycloalkyl comprising one or more N, O or S heteroatoms or 5- to 6-membered heteroaryl comprising one or more N, O or S heteroatoms, wherein each hydrogen atom in alkyl, alkenyl, cycloalkyl, heterocycloalkyl, aryl, or heteroaryl is optionally substituted by halo, hydroxy, C₁₋₄ alkyl, C₁₋₄ alkanol, C₅₋₆ aryl, —OR^{c}, —NR^{c}R^{d}, —CHO, —C(O)R^{c}, —CO₂R^{c}, —C(O)NR^{c}R^{d}, —CN, nitro, or —P(O)OR^{c}OR^{d}; and
each R^{c} and R^{d} is independently H, C₁₋₄ alkyl, C₁₋₄ alkenyl, C₃₋₈ cycloalkyl, 3- to 8-membered heterocyclyl comprising one or more N, O or S heteroatoms, C₅₋₆ aryl, or 5- to 6-membered heteroaryl comprising one or more N, O or S heteroatoms.

39. The compound of claim 38, wherein each R¹ is independently halo, C₁₋₄ alkyl, C₁₋₄ haloalkyl, nitro, —CH═CH₂, —OCH₃, —NR^{a}R^{b}, —CHO, —C(O)R^{a}, —CO₂R^{a}, —C(O)NR^{a}R^{b}, —CN, or cyclopropyl; and R^{a} and R^{b} are each independently H or methyl.

40. The compound of claim 39, wherein
a. each R₁ is independently halo or C₁₋₄ alkyl; and
b. each of R₂ and R₃ is H, halo, C₁₋₄ alkyl, C₁₋₄ haloalkyl.

41. The compound of claim 40, wherein
a. X and Y are each O;
b. q is 0 or 1; and
c. m is 1.

42. The compound of claim 40, wherein the compound is of Formula (IA-1): 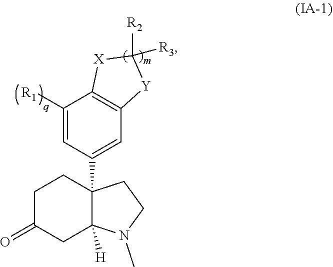
or a pharmaceutically acceptable salt thereof, wherein q is 0 or 1.

43. The compound of claim 40, wherein
a. each of R₂ and R₃ is H, F, or methyl;
b. R₄ is H or methyl; and
c. m is 1.

44. The compound of claim 38, wherein
a. X and Y are each O; or X is O and Y is S; or X is S and Y is O; or X is NR⁴ and Y is O; or X is O and Y is NR⁴;
b. R² and R³ taken together with the atoms to which they are attached combine to form carbonyl, C₃₋₈ cycloalkyl, or 3- to 8-membered heterocyclyl comprising one or more N, O or S heteroatoms; wherein each hydrogen atom in cycloalkyl and heterocyclyl is optionally substituted by halo or OR^{a};
c. R^{a} is H or C₁₋₄ alkyl; and
d. R⁴ is H or methyl.

45. The compound of claim 44, wherein R² and R³ taken together with the atoms to which they are attached combine to form C₃₋₆ cycloalkyl or 3- to 6-membered heterocycloalkyl comprising one or more N, O or S heteroatoms.

46. The compound of claim 44, wherein R² and R³ taken together with the atoms to which they are attached combine to form cyclopropyl, cyclobutyl, oxetane or tetrahydrofuran.

47. The compound of claim 38, wherein
a. X and Y are each O;
b. R² and R³ are each independently halogen, methyl or hydrogen; and
c. q is 0.

48. The compound of claim 38, wherein the compound is of Formula (IA-1): 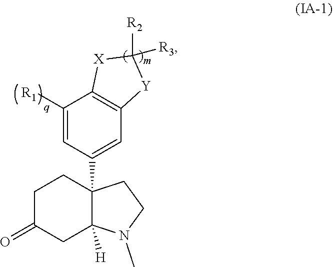
or a pharmaceutically acceptable salt thereof, wherein
X and Y are each O; or X is O and Y is S; or X is S and Y is O; or X is NR⁴ and Y is O; or X is O and Y is NR⁴;
R⁴ is H or methyl;
R¹ is H, halo, —CN, —NO₂, or methyl;
q is 0 or 1;
m is 1; and
R² and R³ are each H, methyl or F; or R² and R³ taken together with the atoms to which they are attached combine to form cyclopropyl, cyclobutyl, oxetane or tetrahydrofuran.

49. The compound of claim 38, wherein the compound is a compound of Formula (IB-1): 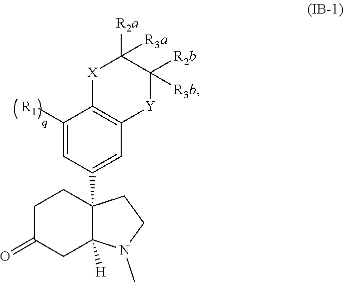
or a pharmaceutically acceptable salt thereof, wherein q is 0 or 1, and
R₂ₐ and R₃ₐ are each independently H or F, and R_{2b} and R_{3b} are each independently H; or R₂ₐ and R₃ₐ are each independently H, and R_{2b}, R_{3b} are each independently H or F; and
R¹ is H, halo, —CN, —NO₂, or methyl.

50. The compound of claim 38, selected from: 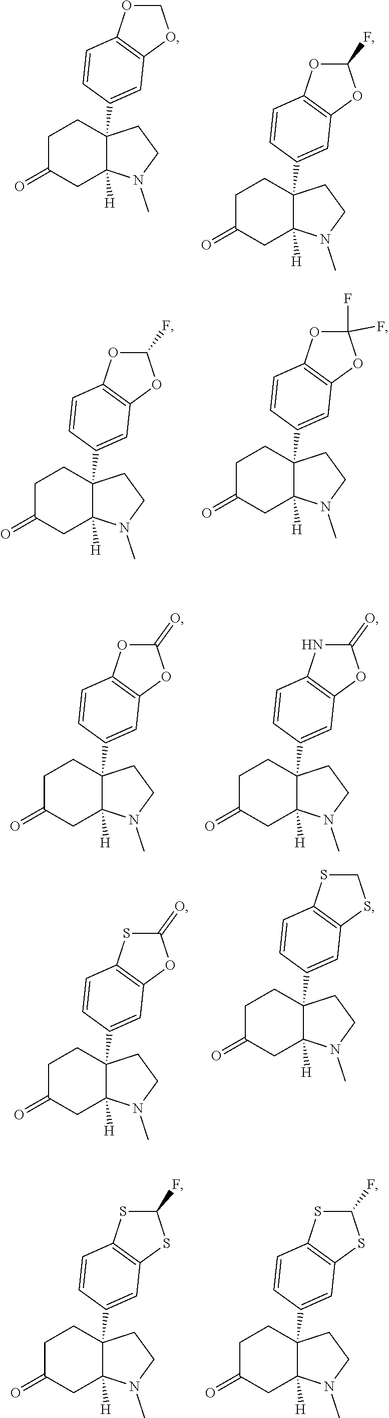 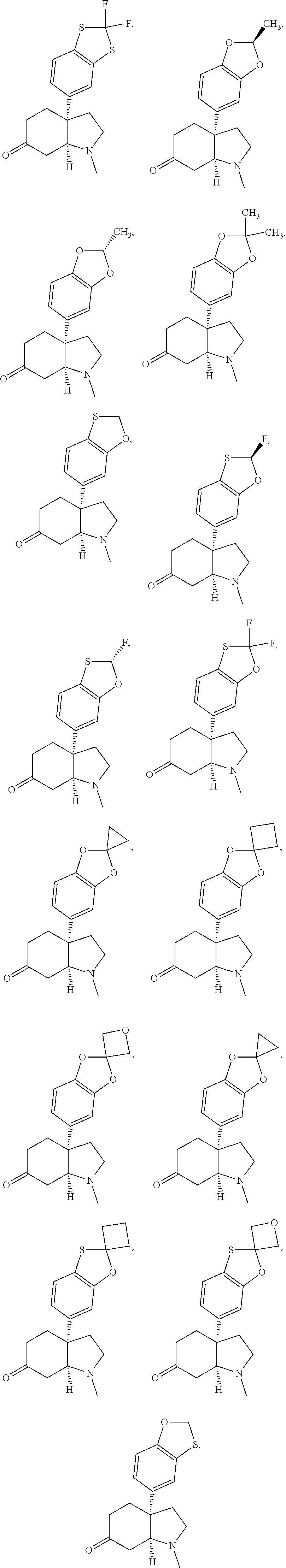 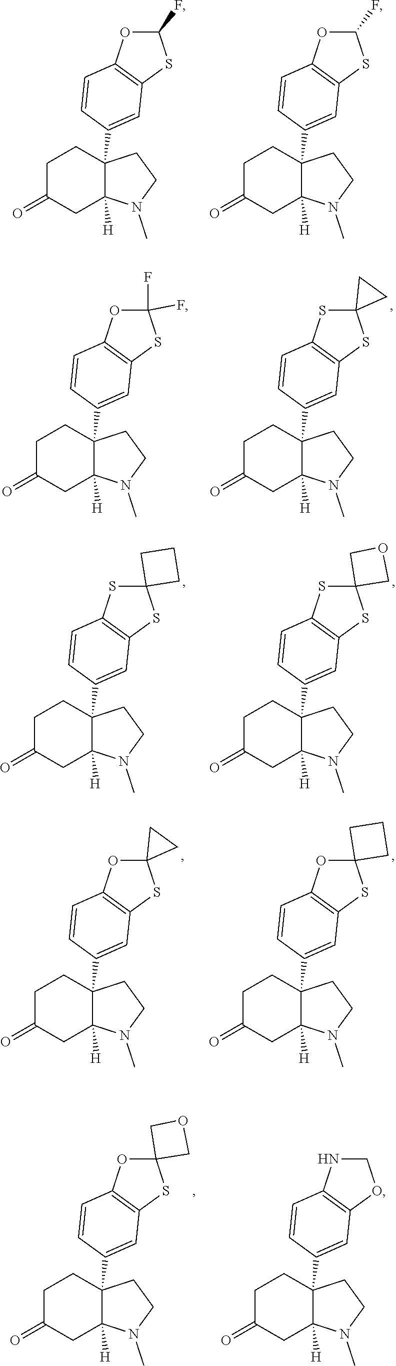 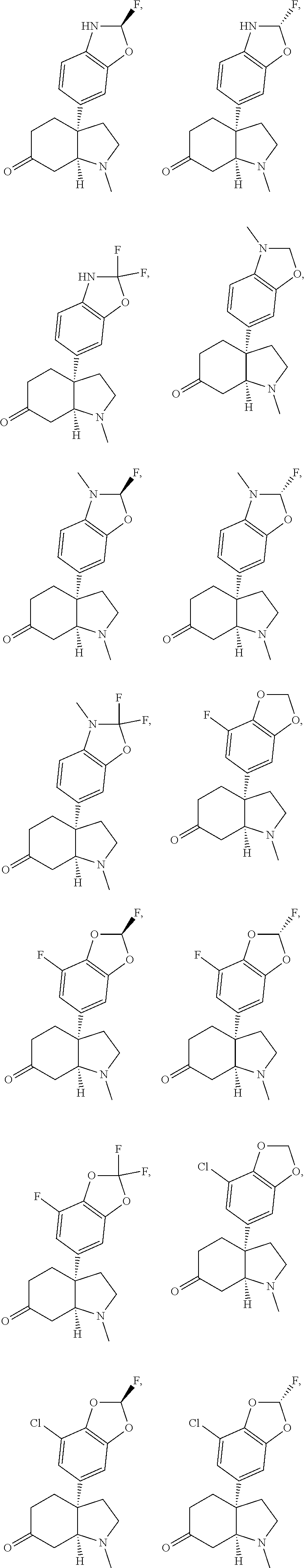 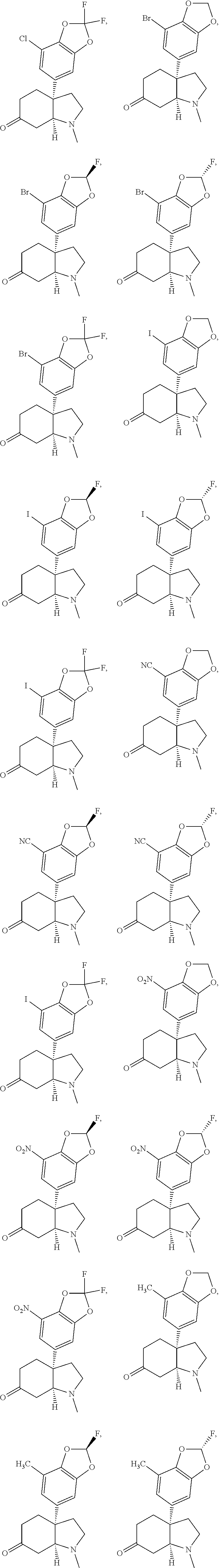 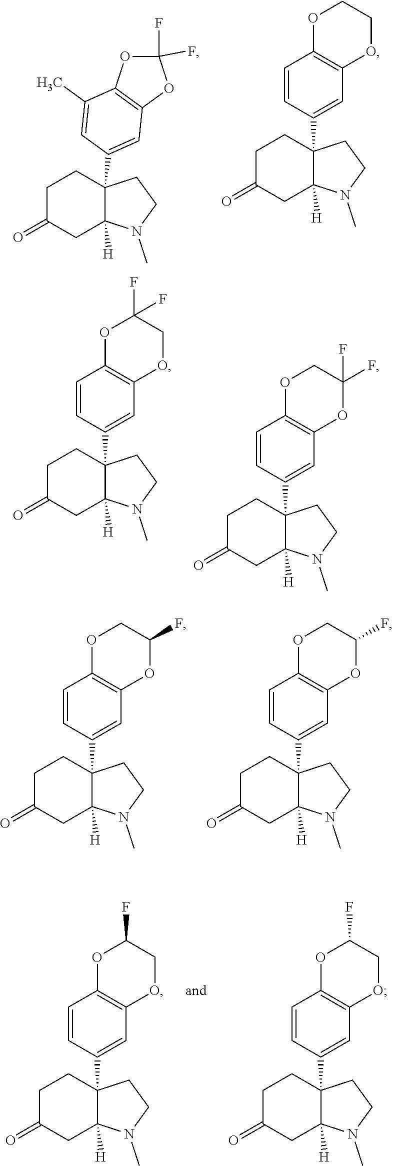
or a pharmaceutically acceptable salt thereof.

51. The compound of claim 38, wherein the compound is 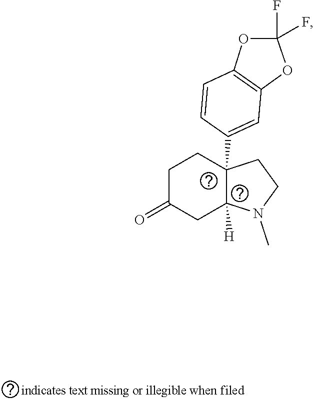 [SMILES: CN1CC[C@]2(c3ccc4c(c3)OC(F)(F)O4)CCC(=O)C[C@H]12]
or a pharmaceutically acceptable salt thereof.

52. The compound of claim 38, wherein the compound is 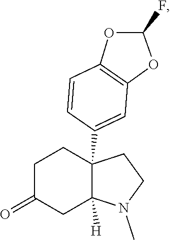 [SMILES: CN1CC[C@]2(c3ccc4c(c3)O[C@H](F)O4)CCC(=O)C[C@H]12]
or a pharmaceutically acceptable salt thereof.

53. The compound of claim 38, wherein the compound is 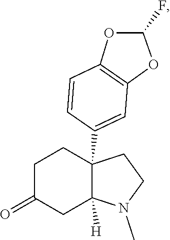 [SMILES: CN1CC[C@]2(c3ccc4c(c3)O[C@@H](F)O4)CCC(=O)C[C@H]12]
or a pharmaceutically acceptable salt thereof.

54. The compound of claim 38, wherein the compound is 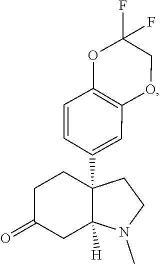 [SMILES: CN1CC[C@]2(c3ccc4c(c3)OCC(F)(F)O4)CCC(=O)C[C@H]12]
or a pharmaceutically acceptable salt thereof.

55. The compound of claim 38, wherein the compound is 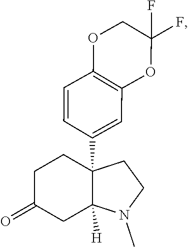 [SMILES: CN1CC[C@]2(c3ccc4c(c3)OC(F)(F)CO4)CCC(=O)C[C@H]12]
or a pharmaceutically acceptable salt thereof.

56. A pharmaceutical composition, comprising a compound of claim 38; and a pharmaceutically acceptable excipient.

57. A method of treating a mental health disorder, comprising administering to a mammal in need thereof an effective amount of a compound of claim 38, or a pharmaceutically acceptable salt thereof.
